(19) United States
(12) Patent Application Publication
(10) Pub. No.: US 2010/0068147 A1
(43) Pub. Date: Mar. 18, 2010

(54) DENGUE DIAGNOSIS AND TREATMENT

(75) Inventors: Martin Lloyd Hibberd, Singapore (SG); Thomas Tolfvenstam, Stockholm (SE); Joshua Fink, Stockholm (SE); Subhash Vasudevan, Singapore (SG); Feng Gu, Singapore (SG)

Correspondence Address: WILMERHALE/NEW YORK, 399 PARK AVENUE, NEW YORK, NY, 10022, US

(73) Assignees: Agency for Science , Technology and Research, Singapore (SG); Novartis AG, Basel (CH)

(21) Appl. No.: 12/444,549

(22) PCT Filed: Oct. 5, 2007
(86) PCT No.: PCT/SG2007/000336

§ 371 Date: Sep. 24, 2009

Related U.S. Application Data
(60) Provisional application No. 60/828,273, filed on Oct. 5, 2006.

(51) Int. Cl. A61K 38/06; C12Q 1/70; A61K 49/00; C12Q 1/37; C12Q 1/00; G01N 33/569; C07K 5/00

(52) U.S. Cl. 424/9.2; 435/5; 435/23; 435/4; 530/331; 514/18; 977/734

(57) ABSTRACT

We describe a method of providing an indication useful in the diagnosis or prognosis of dengue, the method comprising detecting a change in the expression pattern or level of: (a) a ubiquitin-proteasome pathway protein; (b) a interferon-related protein; or (c) an NF-κB-mediated cytokine/chemokine response protein. We also describe a method of identifying a molecule suitable for the treatment or prevention of dengue, the method comprising determining if a candidate molecule is an agonist or antagonist of any one or more of these proteins.

### FIELD

The present invention relates to the fields of medicine, cell biology, molecular biology and genetics. More particularly, the invention relates to a method of diagnosis, prognosis or treating dengue infection in an individual, and/or controlling the replication of dengue virus.

### BACKGROUND

Dengue is the most significant mosquito-born viral disease affecting humans. Up to one third of the world's population is at risk of dengue infection. Infection by dengue virus may result in a spectrum of clinical manifestations. These range from asymptomatic infection through dengue fever (DF) to dengue haemorrhagic fever (DHF) and dengue shock syndrome (DSS).

It has been estimated that there are 50-100 million cases of DF and 250,000 to 500,000 cases of DHF each year {Rigau-Perez, 1998}. Dengue is a small single-stranded RNA virus of the family Flaviviridae comprised of four distinct serotypes (DEN1-4). Its genome consists of a single open reading frame directing the synthesis of a polypeptide which is cleaved by viral and host proteases into ten viral proteins. These include three structural proteins, core (C), envelope (E) and membrane (M), synthesized in precursor form (prM), and seven non-structural (NS) proteins {Rigau-Perez, 1998}.

Despite the seriousness of dengue-related disease, a complete understanding of dengue pathogenesis remains elusive. It has been shown that higher virus titres during the early stages of DF correlates with progression to the more severe DHF {Vaughn, 2000 #708}. However, the lack of proper diagnostics and markers for monitoring the disease progression adds difficulties in predicting the severe outcome. With no vaccine or specific treatment available, a drug which reduced viral replication, and thus prevented the high viral load associated with more severe forms of the disease, represents an attractive option in the fight against dengue. Biomarkers for monitoring the disease course and outcome will also be critical in determining the proper treatment of the disease.

Current methods of diagnosing dengue use either PCR based approaches to look for viral genome (effective but technically difficult and expensive, requiring extensive laboratory facilities); or antibody detection methods to look for host IgG or IgM (that require 3 to 10 days post fever onset to reach detectable levels). Neither method allows rapid, sensitive detection at onset of fever that could be performed in a non-laboratory setting.

There is a need in the art to provide new therapy options for treating dengue infection.

### SUMMARY

This invention is based on the demonstration that inhibition of ubiquitin-proteasome pathway in cells supporting dengue virus infection significantly reduces presence of infectious viral particles released from these cells. Furthermore, we demonstrate that several of the genes coding for proteins active in the ubiquitin-proteasome pathway was specifically up-regulated in cells infected with dengue virus both in-vitro and in human symptomatic infection.

According to a 1^{st} aspect of the present invention, we provide a method of providing an indication useful in the diagnosis or prognosis of dengue, the method comprising detecting a change in the expression pattern or level of: (a) a ubiquitin-proteasome pathway protein; (b) a interferon-related protein; or (c) an NF-κB-mediated cytokine/chemokine response protein.

There is provided, according to a 2^{nd} aspect of the present invention, a method of treatment or prevention of dengue in an individual, the method comprising modulating the level of expression of (a) a ubiquitin-proteasome pathway protein; or (b) a interferon-related protein.

We provide, according to a 3^{rd} aspect, of the present invention, a method of identifying a molecule suitable for the treatment of dengue, the method comprising determining if a candidate molecule is an agonist or antagonist of (a) a ubiquitin-proteasome pathway protein; or (b) a interferon-related protein.

In some embodiments, the candidate molecule is exposed to (a) a ubiquitin-proteasome pathway protein; or (b) a interferon-related protein in order to determine if the candidate molecule is an agonist or antagonist thereof.

As a 4^{th} aspect of the present invention, there is provided use of a polynucleotide encoding: (a) a ubiquitin-proteasome pathway protein; (b) a interferon-related protein; or (c) an NF-κB-mediated cytokine/chemokine response protein for a method as set out above.

We provide, according to a 5^{th} aspect of the present invention, a method for providing an indication useful in the diagnosis or prognosis of dengue, the method comprising detecting a polymorphism in (a) a ubiquitin-proteasome pathway protein; (b) a interferon-related protein; or (c)an NF-κB-mediated cytokine/chemokine response protein, in a sample from the individual.

The present invention, in a 6^{th} aspect, provides a method of identifying an agonist or antagonist of: (a) a ubiquitin-proteasome pathway protein; or (b) a interferon-related protein, the method comprising exposing the candidate molecule to a cell infected with dengue virus and determining an effect on viral function.

In some embodiments, the viral function is selected from the group consisting of: viral titre, viral infectivity, viral replication, viral packaging and viral transcription.

In a 7^{th} aspect of the present invention, there is provided a method of identifying an agonist or antagonist of: (a) a ubiquitin-proteasome pathway protein; or (b) a interferon-related protein, the method comprising administering a candidate molecule to an animal suffering from dengue and determining whether the animal exhibits a decrease or increase in dengue virus replication.

According to an 8^{th} aspect of the present invention, we provide use of an agonist or antagonist of (a) a ubiquitin-proteasome pathway protein; or (b) a interferon-related protein for the preparation of a pharmaceutical composition for the treatment or prevention of dengue in an individual.

We provide, according to a 9^{th} aspect of the invention, a method of down-regulating a dengue viral function in a cell infected with dengue virus, the method comprising modulating the activity of (a) a ubiquitin-proteasome pathway protein; or (b) a interferon-related protein in the cell.

In some embodiments, the viral function is selected from the group consisting of: viral titre, viral infectivity, viral replication, viral packaging and viral transcription.

In some embodiments, the method comprises contacting the virus, the cell or the system with a molecule identified in a method as set out above.

In some embodiments, the polypeptide comprises a ubiquitin-proteasome pathway protein.

The ubiquitin-proteasome pathway protein may be selected from the group consisting of: a ubiquitin specific protease, a ubiquitin-conjugating enzyme, a ubiquitin ligase and a ubiquitin cleavage enzyme.

In some embodiments, the ubiquitin-proteasome pathway protein is selected from the group consisting of: HERC1 (U50078), HERC2 (AF071172), HERC3 (D25215), HERC4 (NM_{—}015601), C17orf27 (AB046774), DTX3L (AK025135), HERC6 (NM_{—}017912), RNF36 (AL360161), ITCH (NM_{—}031483), NEDD4 (NM_{—}006154), UBB (NM_{—}018955), UBE2L6 (NM_{—}004223), UBE2I (NM_{—}003345), Hdm2 (NM_{—}002392), UBE1C (NM_{—}003968), CBL (NM_{—}005188), USP15 (AF106069), USP18 (NM_{—}017414), PSMB9 (NM 002800), UBE2 (NM_{—}003335), UBP43 (NM_{—}017414), HERC5 (NM_{—}016323), ATG7 (NM_{—}006395), DUSP1 (NM 004417.2), DUSP18 (NM_{—}152511.2), DUSP3 (NM_{—}004090.2), DUSP5 (NM_{—}004419.2), EIF3S5 (NM_{—}003754), PPP1R15A (NM_{—}014330.2), PSMB8 (NM_{—}148919), UBE1L (NM_{—}003335), UBE2L6 (NM_{—}004223), UBE2S (NM_{—}014501), UBE2W (NM_{—}018299), USP24 (XM_{—}165973.4) and WWP1 (NM_{—}007013).

The ubiquitin-proteasome pathway protein may comprise ubiquitin specific protease 18 (USP18, GenBank Accession Number: NM_{—}017414) or Ubiquitin-conjugating enzyme E2L (UBE2L6, GenBank Accession Number: NM_{—}004223).

The ubiquitin-proteasome pathway protein inhibitor may comprise MG-132 (Carbobenzoxy-L-leucyl-L-leucyl-L-leucinal) or ALLN (N-Acetyl-Leu-Leu-Nle-CHO).

There is provided, in accordance with a 10^{th} aspect of the present invention, use of MG-132 (Carbobenzoxy-L-leucyl-L-leucyl-L-leucinal) or ALLN (N-Acetyl-Leu-Leu-Nle-CHO) in the preparation of a medicament for the treatment or prevention of dengue in an individual.

The polypeptide may comprise a interferon-mediated protein.

In some embodiments, the interferon-mediated protein is selected from the group consisting of: IFNA1 (NM_{—}024013), IFNB1 (NM_{—}002176), IFNG (NM_{—}000619), ATF3 (NM_{—}004024) MKP-1 (NM_{—}004417, AJ227912), IRF9 (NM_{—}006084), STAT1 (AK022231, NM_{—}007315), G1P2 (NM_{—}005101), G1P3 (NM 002038), IF144 (NM_{—}006417), IFIT1 (NM_{—}001548), IFIT2 (AF026944), IFIT3 (AF026943), ISGF3G (NM_{—}006084), IER3 (NM_{—}003897), IFIT5 (NM_{—}012420), IFRG28 (AJ251832), MDAS (AF095844), SP110 (NM_{—}004510), STAT1 (NM_{—}007315), OAS1 (NM_{—}016816), SOCS1 (NM_{—}003745), ISG15 (NM_{—}005101), TEED (AL080107), OAS3 (NM_{—}006187), IFI44 (NM_{—}006417), OAS2 (NM_{—}002535), MxA (NM_{—}002462), Viperin (AF026941, AF026942), OASL (AF063611), GBP1 (NM_{—}002053), IRF1 (NM_{—}002198), IRF7 (NM 004030), GBP2 (NM_{—}004120), NMI (NM_{—}004688), AIM2 (NM_{—}004833), STAT2 (NM 005419), IFI16 (NM_{—}005531), SLAMF7 (NM_{—}021181), GBP4 (NM_{—}052941) and GBP5 (NM_{—}052942).

The interferon-mediated protein may comprise viperin (GenBank Accession Number: AF026941, AF026942) or interferon alpha (IFN-α, GenBank Accession Number: NM_{—}024013).

As an 11^{th} aspect of the invention, we provide a method of down-regulating a dengue viral function, for example viral titre, viral infectivity, viral replication, viral packaging or viral transcription, in a cell infected with dengue virus, the method comprising up-regulating the activity of viperin (GenBank Accession Number: AF026941, AF026942) in the cell.

The method may further comprise up-regulating the activity of IFN-β (GenBank Accession Number: NM 002176) in the cell.

We provide, according to a 12^{th} aspect of the invention, use of viperin or interferon alpha (IFN-α, GenBank Accession Number: NM_{—}024013), optionally in combination with IFN-β (GenBank Accession Number: NM 002176) in the treatment or alleviation of dengue infection in an individual.

The polypeptide may comprise an NF-κB-mediated cytokine/chemokine response protein.

In some embodiments, the NF-κB-mediated cytokine/chemokine response protein is selected from the group consisting of: COX2 (NM_{—}000963), INOS (NM 000625), IL10 (NM_{—}000572), IL2 (NM_{—}000586), IL6 (NM_{—}000600), IL8 (M17017), RANTES (NM_{—}002985), VEGF (NM_{——}003376), NFKBIB (NM_{——}002503), PAI1 (NM_{—}000602), B2M (NM_{—}004048), NFKBIA (NM_{—}020529), TNFAIP3 (NM_{—}006290), RIG-I (NM_{—}014314), TNF (NM_{—}000594), CCL4 (NM_{—}002984), CCL5 (NM_{—}002985), IL11b (NM_{—}000881), IP-10 (NM_{—}001565), I-TAC (NM_{—}005409), CARD15 (NM 022162), CARD4 (NM_{—}006092), CD14 (NM_{—}000591), CD1A (NM_{—}001763), CD2 (NM_{—}001767), CD22 (NM_{—}001771), CD276 (NM_{—}025240), CD47 (NM 001777), CD59 (NM_{—}000611), CD97 (NM_{—}001784), CCL2 (NM_{—}002982), CCR1 (NM_{—}001295), CCR5 (NM_{—}000579), CCR7 (NM_{—}001838), CCRL2 (NM_{—}003965), CXCL16 (NM_{—}022059), IL1RN (NM_{—}173842), IL10RB (NM_{—}000628), IL13RA1 (NM_{—}001560), IL16 (NM_{—}004513), IL18 (NM 001562), IL18RAP (NM_{—}003853), IL4R (NM_{—}000418), IL8RA (NM_{—}000634), IL8RB (NM_{—}001557), PF4 (NM_{—}002619), PBEF1 (NM_{—}182790), TNFSF10 (NM_{—}003810), TNFRSF1A (NM_{—}001065), TNFRSF1B (NM_{—}001066), TNFRSF25, (NM_{—}148970), TNFRSF7 (NM_{—}001242), TNFAIP2 (NM_{—}006291) and TNFAIP8 (NM_{—}014350).

The NF-κB-mediated cytokine/chemokine response protein may comprise IP-10 (GenBank Accession Number: NM_{—}001565). The NF-κB-mediated cytokine/chemokine response protein may comprise I-TAC (GenBank Accession Number: NM_{—}005409).

We provide, according to a 12^{th} aspect of the invention, a method of providing an indication useful in the diagnosis or prognosis of dengue, the method comprising detecting a change in the expression pattern or level of IP-10 (GenBank Accession Number: NM_{—}001565) or I-TAC (GenBank Accession Number: NM_{—}005409), or both.

We provide, according to a 13^{th} aspect of the invention, a method of providing an indication useful in the diagnosis or prognosis of dengue, the method comprising detecting a change in the expression pattern or level of any one or more of the following: interferon alpha (IFN-α, GenBank Accession Number: NM 024013), IP-10 (GenBank Accession Number: NM_{—}001565) or I-TAC (GenBank Accession Number: NM_{—}005409).

We provide, according to a 14^{th} aspect of the invention, a kit for diagnosis or prognosis of dengue, the kit comprising means for the detection of a change in the expression pattern or level of any one or more of the following: (a) a ubiquitin-proteasome pathway protein; (b) a interferon-related protein; or (c) an NF-κB-mediated cytokine/chemokine response protein, together with instructions for use.

We provide, according to a 15^{th} aspect of the invention, a kit for treatment or prevention of dengue in an individual, the kit comprising means for modulating the level of expression of: (a) a ubiquitin-proteasome pathway protein; or (b) a interferon-related protein, together with instructions for use.

The kit may comprise MG-132 (Carbobenzoxy-L-leucyl-L-leucyl-L-leucinal) or ALLN (N-Acetyl-Leu-Leu-Nle-CHO) or both.

The kit may further comprise any one or more of the following: P4-PMO compounds 5′SL and 3′CS, any of the fullerenes described in U.S. Pat. No. 6,777,445 and Helioxanthin and/or an analogue thereof (U.S. Pat. No. 6,306,899)

We provide, according to a 16^{th} aspect of the invention, a molecule identified by a screening method as provided.

We provide, according to a 17^{th} aspect of the invention, an agonist or antagonist of (a) a ubiquitin-proteasome pathway protein; or (b) a interferon-related protein identified by a screening method as provided.

We provide, according to a 18^{th} aspect of the invention, use of such a molecule, agonist or antagonist for the treatment, prevention or diagnosis or prognosis of dengue in an individual.

We provide, according to a 19^{th} aspect of the invention, MG-132 (Carbobenzoxy-L-leucyl-L-leucyl-L-leucinal) for use in a method of treatment, prevention or diagnosis or prognosis of dengue in an individual.

We provide, according to a 20^{th} aspect of the invention, ALLN (N-Acetyl-Leu-Leu-Nle-CHO) for use in a method of treatment, prevention or diagnosis or prognosis of dengue in an individual.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies : A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1. The ubiquitin-proteasome pathway.

FIG. 2. Structure of MG-132, a potent, reversible, and cell-permeable proteasome inhibitor.

FIG. 3A, FIG. 3B and FIG. 3C. Plaque-assay showing plaque-forming units (pfu) after infection of HepG2 cells with TSV01 virus in the presence of ubiquitin-inhibiting compounds and DMSO control. *) indicates p<0.05

FIG. 4A and FIG. 4B are graphs showing that ubiquitin-proteasome inhibitors inhibits dengue virus replication in HepG2 cells.

FIG. 5. a) Heat map of microarray expression profile of HepG2 transcripts differentially expressed in response to dengue virus TSV01 and heat inactivated TSV01, selected by SAM analysis. Details on gene name, gene function, SAM statistics, and fold change for all 132 transcripts are given in supplementary table 1. Mean relative expression of dye swap results in comparison to universal reference RNA. Fold changes are represented by color, red as upregulated and green downregulated. Time course (3, 6, 12, 24, 48, 72 hours post infection) and replicate (0.1, 0.2, 0.3) is indicated for TSV01 (V) and Heat inactivated virus (H). and genes are grouped by pathway (selected in 1b), then clustered by expression profile. 1b) Pathway analysis of SAM selected transcripts using Panther biological processes revealed two highly significant gene clusters.

FIG. 6. Common genes up-regulated in all systems, as shown by TLDA, map to specific pathways. (a-c) The 50 genes in common across the three systems were grouped according to their biological functions to three specific pathways; interferon-mediated genes(a), genes of the ubiquitin-proteasome system (b) and NF-kappaB-mediated or cytokine/mediator genes (c). The mean fold increase of these common genes in each system, HepG2 cells, A549 cells and patient samples (Patients), were combined to give a pooled mean which was plotted (mean±s.e.m., where n=3-5 and “*” indicates genes shown on the map in FIG. 2d. Genes from the same TLDA along the same pathways but not consistently up-regulated in all three systems are listed in table form with “+” indicating the gene was significantly upregulated and “−” indicating it was not. (d) These fifty common genes were mapped by direct interactions using the MetaCoreprogram. Arrowhead indicates directional interaction. Green=positive, red=negative, grey=neutral or unknown. Blue symbols indicate protein. Green ones indicate receptor ligand. Red ones indicate transcription factors. Yellow ones are protease and orange ones are kinase and phosphatase.

FIG. 7. Functional effects of components of each pathway following dengue infection and ondengue replication. (a, b) A549 and HepG2 cells were infected with dengue virus TSV01 (MOI 10) or heat inactivated virus (HI-TSV01;MOI10) for 72 hrs before cell culture supernatants were collected and assayed for the release of chemokines (a) IP-10 and (b) I-TAC by ELISA. Results are expressed as mean±s.e.m. where n=3-5. P values less than 0.05, as determined by students t-test, are shown comparing virus infection to the controls at each time point. (c, d) Plasma samples from dengue patients at Day 1, Day 3, Day 21 and from patients with non-dengue associated fever patients (at Day 1) were assayed for the presence of (c) IP-10 and (d) I-TAC by ELISA. Results are expressed as a scatter plot with a bar indicating the mean, where n=10 (see main text for statistical comparisons). (e, f) Wild type (wt) and viperin over expressing (Vip)A549 cells, either with (e) or without (f) 12 hrs pre-treatment with IFN-β (500 U/ml), were infected with dengue virus TSV01 (MOI 1) for 48 hrs. Plaque forming units/ml, as determined by plaque assay, are expressed as mean±s.e.m. where n=3. P values, as determined by students t-test, are shown comparing wild type cells to viperin cells. (g) HepG2 cells were incubated with MG-132 (0.04 μM and 0.4 μM in DMSO) and ALLN (1 μM and 10 μM in DMSO) and with DMSO alone for 2 hrs prior to infection with dengue virus TSV01 (MOI 10) for 48 hrs before cell culture supernatants were assayed for dengue virus by plaque assay. Results are expressed as the mean percentage of the highest number of pfu/ml (DMSO alone)±s.e.m. (n=3). P values less than 0.05, as determined by students t-test, are shown comparing each treatment to DMSO alone.

FIG. 8. Infection of HepG2 cells with dengue virus. (a) HepG2 cells were infected with dengue virus TSV01 (MOI 10) for 3, 6, 12, 24, 48 and 72 hrs. Cell culture supernatants were collected and assayed for dengue virus by plaque assay. Plaque forming units per ml are expressed on a log scale as mean±s.e.m. where n=4. (b) HepG2 cells were infected with dengue virus TSV01 for the indicated timepoints (closed bars) or with media alone (open bars). Cells were then stained with the Alexa 647-conjugated 4G2 antibody against dengue E-protein and analysis by FACS. Percentage of cells staining positive for dengue virus are expressed as mean±s.e.m. where n=3. (c) HepG2 cells were infected with dengue virus TSV01 (MOI10) for 3, 6, 12, 24, 48 and 72 hrs. Viral RNA were extracted and quantified by real-time PCR. Results are expressed as an inversed ratio to the Cp values (number of cycle) and are representative of three distinct experiments.

### DETAILED DESCRIPTION

In this invention, we have identified a number of human serum proteins, in particular those of the NFkB initiated chemokine pathway that would be suitable targets for a diagnostic or prognostic test for Dengue. The proteins, either singular or in combination together, or in combination with viral proteins such as NS1, are able to detect the presence of a dengue infection and form the basis of our dengue detection invention.

We have also identified two host-response to dengue infection pathways, relating to interferon signalling, (Ubiquitin and Interferon pathway members) that prevent dengue virus replication and are thus novel, human, drug targets for controlling dengue viral replication and thus dengue disease.

Thus, we have demonstrated that dengue infection results in the elevated expression of a number of genes belonging to the following groups: (a) a ubiquitin-proteasome pathway protein; (b) a interferon-related protein; or (c) an NF-κB-mediated cytokine/chemokine response protein.

Accordingly, detection of expression of any one or more genes as described in this document can be used to diagnose, or provide an indication useful in the diagnosis of, dengue infection. Where the term “diagnosis” is used in this document, it should be taken to include prognosis as well as diagnosis.

In particular, detection of expression of any one or more genes as described in this document can be used to detect dengue infection, to distinguish dengue infection from other diseases which may have the same or similar symptoms, and to predict the severity of disease.

We have established that it is possible to distinguish patients having fever as suffering from dengue from patients having fever, but who do not have dengue.

For example, the level of expression or activity of interferon alpha (IFN-α, GenBank Accession Number: NM_{—}024013) may be detected to detect dengue. A serum level of 389 ng/microlitre of interferon alpha may be used for example as a cut-off point. Thus, if a patient has a serum level of more than about 389 ng/microlitre of interferon alpha, then he is likely be actually suffering from dengue (rather than having a fever arising from other, non-dengue, causes, i.e., a non-dengue febrile illness). It will be appreciated that the level of 389 ng/microlitre of interferon alpha is not an absolute figure, and that it is possible to use other levels, e.g, in the range of 350-450 ng/microlitre of interferon alpha as cut-offs, but with perhaps a lower level of accuracy.

In addition, we find that the genes may be used as markers for the severity of dengue disease. Accordingly they may be used for example to assess whether it is likely that a patient needs further treatment, e.g., hospitalisation.

For example, the level of expression or activity of IP-10 (GenBank Accession Number: NM_{—}001565) may be detected as an indicator of the likelihood or severity of the disease. A serum level of 1697.9 ng/microlitre of IP-10 may be used for example as a cut-off point. Thus, if a patient has a serum level of more than about 1697.9 ng/microlitre of IP-10, then he is likely to develop dengue severe disease. It will be appreciated that the level of 1697.9 ng/microlitre of IP-10 is not an absolute figure, and that it is possible to use other levels, e.g, in the range of 1300-2100 ng/microlitre of IP-10 as cut-offs, but with perhaps a lower level of accuracy.

The expression or activity of the genes and proteins described here may be detected together with other indicators of dengue disease, for example, body temperature, pulse, blood pressure, blood cell count, haematocrit, haemoglobin levels, etc.

Furthermore, we find that these genes are activated in patients with dengue disease and that inhibiting these genes using compounds greatly reduces virus production in an in vitro model. We therefore provide for the use of any of these genes or corresponding proteins in the treatment or prevention of dengue. For example, activity or expression of the genes may be regulated for treating or preventing dengue. The genes or proteins may also be used as targets for drug development. We therefore provide for screens for molecules which bind to, agonise or antagonise any of these genes in the three pathways. Such screens may be used to identify molecules suitable for the treatment or prevention of dengue.

Therefore, accurately targeting the genes in these three groups (for example, ubiquitin pathway genes or the aspects of the ubiquitin pathway they represent) using small molecules, or other drug mechanisms, to reduce their activation is likely to provide a novel therapy for dengue disease.

This invention shows the mechanisms to enable early, rapid and easy diagnosis and prognosis of dengue infection; together with novel human drug targets that would prevent dengue disease.

According to the methods and compositions described here, any protein or activity involved in the following groups (a) a ubiquitin-proteasome pathway protein; (b) a interferon-related protein; or (c) an NF-κB-mediated cytokine/chemokine response protein may be modulated in order to reduce dengue viral function, which may be for the treatment or alleviation of dengue in an individual. Specifically, dengue may be treated or prevented by modulating the level of expression of, or the activity of, or both of any of these proteins. Thus, for example, a compound capable of modulating the ubiquitin-proteasome pathway may be used as a treatment for dengue.

Any component of the relevant pathway in a cell may be modulated, in protein level, in activity or in expression. In some embodiments, the activity of a relevant protein is down-regulated to disrupt viral function or to treat dengue infection.

The inhibitor or inhibitors of these genes may be used in combination with any agent which is known or suspected to be efficacious in treating or alleviating dengue. Examples include the P4-PMO compounds 5′SL and 3′CS (targeting the 5′-terminal nucleotides and the 3′ cyclization sequence region, respectively) as described in Kinney et al., 2005, Inhibition of dengue virus serotypes 1 to 4 in vero cell cultures with morpholino oligomers, J Virol. 79(8), 5116-28. Other examples include the fullerenes described in U.S. Pat. No. 6,777,445. U.S. Pat. No. 6,306,899 describes methods of inhibition and treatment of Flavivirus including Dengue virus by Helioxanthin and its analogs, and such compounds may also be used in combination with ubiquitin-proteasome pathway protein inhibitors as described herein for the treatment or alleviation of dengue.

The inhibitors, agonists, antagonists, etc for the treatment, prevention, alleviation and/or diagnosis of dengue may be packaged in a kit.

Such a kit may comprise means for the detection of a change in the expression pattern or level of any one or more of the following: (a) a ubiquitin-proteasome pathway protein; (b) a interferon-related protein; or (c) an NF-κB-mediated cytokine/chemokine response protein, together with instructions for use and may be suitable for detection, diagnosis or prognosis of dengue.

A further example of a kit may comprise means for modulating the level of expression of: (a) a ubiquitin-proteasome pathway protein; or (b) a interferon-related protein, together with instructions for use. Such a kit may be useful for treatment or prevention of dengue in an individual.

In particular, the kit may comprise MG-132 (Carbobenzoxy-L-leucyl-L-leucyl-L-leucinal) or ALLN (N-Acetyl-Leu-Leu-Nle-CHO) or both. These are described in detail later in this document.

It will also be appreciated that compounds which are equivalent to these molecules may be used in place of, or in addition to MG-132 and/or ALLN, for treatment or prevention of dengue, and for use in the kits. In particular, we provide for the use of compounds similar in structure or functionally equivalent to MG-132 and/or ALLN for such purposes. These include in particular chemical derivatives of MG-132 and/or ALLN, chemical modifications of MG-132 and/or ALLN, substituted MG-132 and/or ALLN, pharmaceutically acceptable salts of MG-132 and/or ALLN, polymorphic forms of MG-132 and/or ALLN, isotopic variations of MG-132 and/or ALLN, prodrugs of MG-132 and/or ALLN, pro-moieties of MG-132 and/or ALLN and salts of MG-132 and/or ALLN, etc. Such equivalents of MG-132 and/or ALLN are described in detail in a separate section below.

The kit may further comprise any one or more of the following: P4-PMO compounds 5′SL and 3′CS, any of the fullerenes described in U.S. Pat. No. 6,777,445 and Helioxanthin and/or an analogue thereof (U.S. Pat. No. 6,306,899)

### Dengue

For the purposes of this description, the terms “dengue”, “dengue fever” and “dengue hemorrhagic fever” should be considered synonymous.

Dengue and dengue hemorrhagic fever (DHF) are acute febrile diseases, found in the tropics, with a geographical spread similar to malaria. Caused by one of four closely related virus serotypes of the genus Flavivirus, family Flaviviridae, each serotype is sufficiently different that there is no cross-protection and epidemics caused by multiple serotypes (hyperendemicity) can occur. Dengue is transmitted to humans by the mosquito Aedes aegypti (rarely Aedes albopictus).

Signs and Symptoms

The disease is manifested by a sudden onset of fever, with severe headache, joint and muscular pains (myalgias and arthralgias—severe pain gives it the name break-bone fever) and rashes; the dengue rash is characteristically bright red petechia and usually appears first on the lower limbs and the chest—in some patients, it spreads to cover most of the body. There may also be gastritis with some combination of associated abdominal pain, nausea, vomiting or diarrhoea.

Some cases develop much milder symptoms, which can, when no rash is present, be misdiagnosed as a flu or other viral infection. Thus, travellers from tropical areas may inadvertently pass on dengue in their home countries, having not being properly diagnosed at the height of their illness. Patients with dengue can only pass on the infection through mosquitoes or blood products while they are still febrile.

The classic dengue fever lasts about six to seven days, with a smaller peak of fever at the trailing end of the fever (the so-called “biphasic pattern”). Clinically, the platelet count will drop until the patient's temperature is normal.

Cases of DHF also show higher fever, haemorrhagic phenomena, thrombocytopenia and haemoconcentration. A small proportion of cases leads to dengue shock syndrome (DSS) which has a high mortality rate.

Diagnosis

The diagnosis of dengue is usually made clinically. The classic picture is high fever with no localising source of infection, a petechial rash with thrombocytopenia and relative leukopenia.

Serology and PCR (polymerase chain reaction) studies are available to confirm the diagnosis of dengue if clinically indicated.

Treatment

The mainstay of treatment is supportive therapy. The patient is encouraged to keep up oral intake, especially of oral fluids. If the patient is unable to maintain oral intake, supplementation with intravenous fluids may be necessary to prevent dehydration and significant hemoconcentration. A platelet transfusion is indicated if the platelet level drops significantly.

Prevention

There is no commercially available vaccine for the dengue flavivirus. However, one of the many ongoing vaccine development programs is the Pediatric Dengue Vaccine Initiative (PDVI [1]) which was set up in 2003 with the aim of accelerating the development and introduction of dengue vaccine(s) that are affordable and accessible to poor children in endemic countries.

Primary prevention of dengue mainly resides in eliminating or reducing the mosquito vector for dengue. Initiatives to eradicate pools of standing water (such as in flowerpots) have proven useful in controlling mosquito borne diseases. Promising new techniques have been recently reported from Oxford University on rendering the Aedes mosquito pest sterile.

Personal prevention consists of the use of mosquito nets, repellents and avoiding endemic areas.

[The foregoing description is adapted from Wikipedia contributors (2006). Dengue fever. Wikipedia, The Free Encyclopedia. Retrieved 17:08, Mar. 8, 2006 from http://en.wikipedia.org/w/index.php?title=Dengue_fever&oldid=42596229]

### Assays for Dengue

Dengue infection may be assayed by a number of methods, including a method of plaque assay in some embodiments.

Plaque Assay for Dengue

Confluent monolayers of Vero cells are grown in Iscove's medium (HyClone, Logan, Utah) supplemented with 9% heat-inactivated fetal bovine serum (FBS) (HyClone), sodium bicarbonate (0.75 g/liter), penicillin G (100 U/ml), and streptomycin sulfate (100 μg/ml) (indicated as Iscove-9% FBS medium) in 12-well plates at 37° C. and 5% CO2. Media containing 4.7% FBS (Iscove-4.7% FBS) or lacking FBS (Iscove-0% FBS) are also used in this study.

Vero cells are seeded into 12-well plates at 5.0 to 5.5 log10 cells per well. Viral infection is performed by aspirating the growth medium from freshly confluent Vero cell cultures, washing the cells sheets twice with 2 ml of Iscove-0% FBS medium, and adding 100 μl of Iscove-0% FBS medium containing dengue virus to deliver a multiplicity of infection (MOI) of 1.0 or 2.0 PFU/cell. Following adsorption of virus for 2 h at 37° C. with 5% CO2, the viral inocula are aspirated, the cell sheets are rinsed three times each with 2 ml of PBS, and 1.0 ml of Iscove-0% FBS medium containing the appropriate concentration of P4-PMO is added, followed by incubation of the plates at 37° C. with 5% CO2. Except where stated, the replacement media are not changed again for the duration of the growth curve experiment. Controls for these experiments include untreated cells.

At various time intervals, a 20-μl aliquot of medium is removed from each virus-infected well, diluted 1:16 or 1:32 in freezing medium (Iscove-35% FBS medium), and stored at −80° C. until plaque titration.

Plaque titrations are performed under agarose overlay in Vero cell monolayers grown in six-well plates as described previously (Butrape et al 2000. J. Virol. 74:3011-3019 and Miller and Mitchell, 1986, Am. J. Trop. Med. Hyg. 35:1302-1309). In each viral growth curve, the sensitivity limit of plaque titration is indicated by a horizontal line at 1.9 or 2.2 log10 PFU/ml, which resulted from plating 200 μl of the 1:16 or 1:32 dilution of harvested virus in the first well of the six-well plate, respectively.

Cytotoxicity Assay

Cytotoxicity was monitored using fluorescein diacetate (FDA) as previously described (Zhang et al 2006).

Other Dengue Assays

Payne et al., 2006, J Virol Methods. 27 describes a method for the quantitation of flaviviruses by fluorescent focus assay. Such an assay may be used instead of, or in combination with, a standard plaque assay. In summary, the assay comprises the following:

Vero cells are plated in 8-well chamber slides, and infected with 10-fold serial dilutions of virus. About 1-3 days after infection, cells are fixed, incubated with specific monoclonal antibody, and stained with a secondary antibody labeled with a fluorescent tag. Fluorescent foci of infection are observed and counted using a fluorescence microscope, and viral titers are calculated as fluorescent focus units (FFU) per ml. The optimal time for performing the fluorescent focus assay (FFA) on Vero cells was 24 h for Dengue virus serotypes compared to up to 11 days for a standard Vero cell plaque assay

Other assays which may be used include those described in U.S. Pat. No. 6,855,521, a serotype and dengue group specific flurogenic probe based PCR (TaqMan) assay against the respective C and NS5 genomic and 3′ non-coding regions of dengue virus, and U.S. Pat. No. 6,793,488 which describes a Flavivirus detection and quantification assay

### Ubiquitin-Proteasome Pathway

Ubiquitinylation is an important regulatory tool that controls the concentration of key signalling proteins, such as those involved in cell cycle control, as well as removing misfolded, damaged or mutant proteins that could be harmful to the cell.

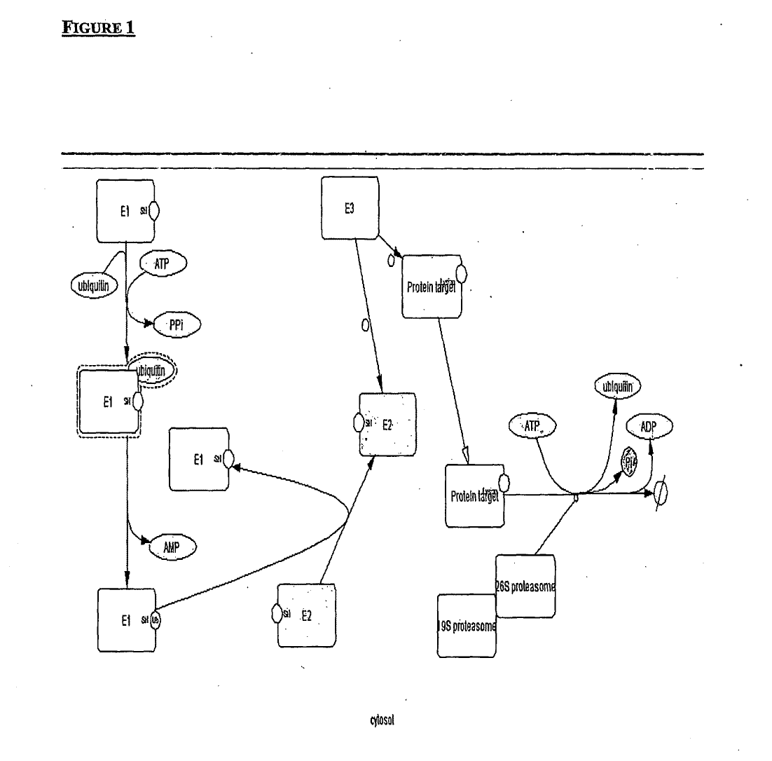

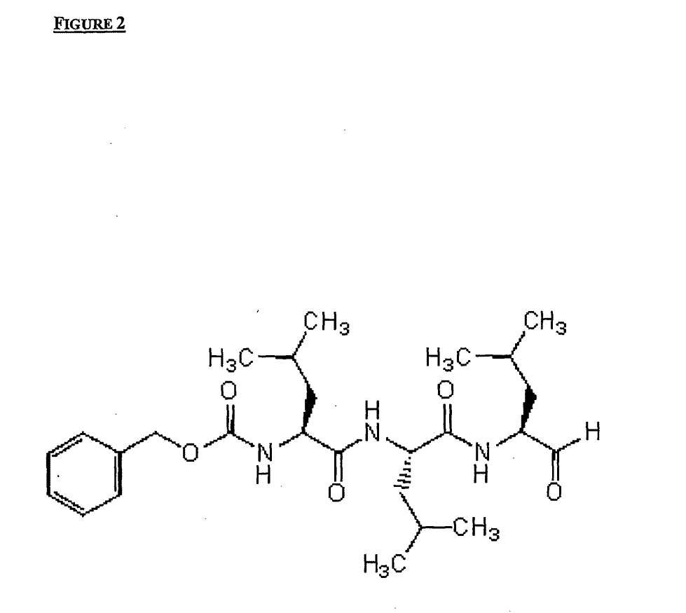

The term “ubiquitin-proteasome pathway” should be taken to refer to the cellular pathway which is responsible for the ubiquitination of substrates for targeting for degradation, as described below and as illustrated in FIG. 1.

Ubiquitin is a protein of 76 amino acid residues, found in all eukaryotic cells and whose sequence is extremely well conserved from protozoan to vertebrates. Ubiquitin acts through its post-translational attachment (ubiquitinylation) to other proteins, where these modifications alter the function, location or trafficking of the protein, or targets it for destruction by the 26S proteasome (Burger and Seth (2004) Eur J Cancer. 40(15):2217-29).

The terminal glycine in the C-terminal 4-residue tail of ubiquitin can form an isopeptide bond with a lysine residue in the target protein, or with a lysine in another ubiquitin molecule to form a ubiquitin chain that attaches itself to a target protein. Ubiquitin has seven lysine residues, any one of which can be used to link ubiquitin molecules together, resulting in different structures that alter the target protein in different ways.

It appears that Lys(11)-, Lys(29) and Lys(48)-linked poly-ubiquitin chains target the protein to the proteasome for degradation, while mono-ubiquitinylated and Lys(6)- or Lys(63)-linked poly-ubiquitin chains signal reversible modifications in protein activity, location or trafficking (Passmore and Barford (2004) Biochem J. 379(Pt 3):513-25). For example, Lys(63)-linked poly-ubiquitinylation is known to be involved in DNA damage tolerance, inflammatory response, protein trafficking and signal transduction through kinase activation (Pickart and Fushman (2004) Curr Opin Chem Biol. 8(6):610-6). In addition, the length of the ubiquitin chain alters the fate of the target protein. Regulatory proteins such as transcription factors and histones are frequent targets of ubquitinylation (de Napoles et al., 2004. Dev Cell 7(5):663-76).

Ubiquitinylation is an ATP-dependent process that involves the action of at least three enzymes: a ubiquitin-activating enzyme (E1), a ubiquitin-conjugating enzyme (E2), and a ubiquitin ligase (E3), which work sequentially in a cascade. There are many different E3 ligases, which are responsible for the type of ubiquitin chain formed, the specificity of the target protein, and the regulation of the ubiquitinylation process (Hatakeyama and Nakayama (2003) Biochem Biophys Res Commun. 302(4):635-45).

Specifically, the process of ubiquitination typically comprises the following steps:

1. Activation of ubiquitin—Ubiquitin is activated in a two-step reaction by an E1 ubiquitin-activating enzyme in a process requiring ATP as an energy source. The initial step involves production of an ubiquitin-adenylate intermediate. The second step transfers ubiquitin to the E1 active site cysteine residue, with release of AMP. This step results in a thioester linkage between the C-terminal carboxyl group of ubiquitin and the E1 cysteine sulfhydryl group.

2. Transfer of ubiquitin from E1 to the active site cysteine of an ubiquitin-conjugating enzyme E2 via a trans(thio)esterification reaction.

3. The final step of the ubiquitylation Cascade generally requires the activity of an E3 ubiquitin-protein ligase (often termed simply ubiquitin ligase). E3 enzymes function as the substrate recognition modules of the system and are capable of interaction with both E2 and substrate. E3 enzymes possess one of two domains: * The HECT (Homologous to the E6-AP Carboxyl Terminus) domain; * The RING domain (or the closely related U-box domain).

Transfer can occur in two ways: * Directly from E2, catalysed by RING domain E3s; * Via an E3 enzyme, catalysed by HECT domain E3s. In this case, a covalent E3-ubiquitin intermediate is formed prior to transfer of ubiquitin to the substrate protein.

Finally, the marked protein is digested in the 26S-proteasome (see below) into small peptides (usually 6-7 amino acid residues in length). Ubiquitin moieties are cleaved off the protein by the proteasome and are recycled for further use.

In some embodiments, the activity of an E1 ubiquitin-activating enzyme is down-regulated. In other embodiments, the activity of an E2 ubiquitin-conjugating enzyme is down-regulated. In yet other embodiments, the activity of an E3 ubiquitin-protein ligase is down-regulated.

In some embodiments, a ubiquitin specific protease, a ubiquitin-conjugating enzyme, a ubiquitin ligase or a ubiquitin cleavage enzyme are modulated. In advantageous embodiments, the ubiquitin-proteasome pathway protein is selected from the group consisting of: G1P2 (ISG15 IFI15, NM_{—}005101), HERC5 (E3 Ub ligase, GenBank Accession Number: NM_{—}016323) and USP18 (ISG15 cleavage, GenBank Accession Number: NM_{—}017414.2). The ubiquitin-proteasome pathway protein may also comprise ubiquitin specific protease 18 (USP18, GenBank Accession Number: NM_{—}017414) or Ubiquitin-conjugating enzyme E2L (UBE2L6, GenBank Accession Number: NM_{—}004223).

Ubiquitin-Proteasome Pathway Protein

By the term “ubiquitin-proteasome pathway protein”, we mean any of the genes involved in the ubiquitin-proteasome pathway. In some embodiments, the ubiquitin-proteasome pathway protein is a protein set out in Table D1 below.

TABLE D1
Ubiquitin-proteasome Pathway Genes

| | GenBank |
|---|---|
| Gene | Accession Number |
| ATG7 | NM_006395 |
| C17orf27 | AB046774 |
| CBL | NM_005188 |
| DTX3L | AK025135 |
| DUSP1 | NM_004417.2 |
| DUSP18 | NM_152511.2 |
| DUSP3 | NM_004090.2 |
| DUSP5 | NM_004419.2 |
| EIF3S5 | NM_003754 |
| Hdm2 | NM_002392 |
| HERC1 | U50078 |
| HERC2 | AF071172 |
| HERC3 | D25215 |
| HERC3 | D25215 |
| HERC4 | NM_015601 |
| HERC5 | NM_016323 |
| HERC6 | NM_017912 |
| ITCH | NM_031483 |
| NEDD4 | NM_006154 |
| PPP1R15A | NM_014330.2 |
| PSMB8 | NM_148919 |
| PSMB9 | NM_002800 |
| RNF36 | AL360161 |
| UBB | NM_018955 |
| UBE1C | NM_003968 |
| UBE1L | NM_003335 |
| UBE2 | NM 003335 |
| UBE2I | NM_003345 |
| UBE2L6 | NM_004223 |
| UBE2L6 | NM_004223 |
| UBE2S | NM_014501 |
| UBE2W | NM_018299 |
| UBP43 | NM_017414 |
| USP15 | AF106069 |
| USP18 | NM_017414 |
| USP24 | XM_165973.4 |
| WWP1 | NM_007013 |

### Proteasome

A multi-component enzymatic complex known as the 26S proteasome hydrolyses the ubiquitinated proteins.

The proteolytic core of this complex, the 20S proteasome, contains multiple peptidase activities. This core is composed of 28 subunits arranged in four heptameric, tightly stacked, rings (α7, β7, β7, α7) to form a cylindrical structure. The α-subunits make up the two outer rings and the β-subunits the two inner rings of the stack. The entrance to the active site of the complex is guarded by the α-subunits that allow access only for the unfolded and extended polypeptides. The regulatory unit of the 26S proteasome is known as the 19S particle consisting of about 17 subunits that include ATPases, a de-ubiquitinating enzyme, and polyubiquitin-binding subunits.

For the purposes of this document, the term “ubiquitin-proteasome pathway protein” should be taken to include reference to the proteasome, and a “ubiquitin-proteasome pathway protein inhibitor” therefore includes anything that is capable of inhibiting any of the activities of the proteasome, for example as described below.

### Proteasome Inhibitor

In advantageous embodiments, the ubiquitin-proteasome pathway protein inhibitor comprises a compound which inhibits an activity of the proteasome, i.e., a proteasome inhibitor compound.

The ubiquitin proteasome comprises a number peptidase activities, and the proteasome inhibitor may inhibit any one or more of these activities. The peptidase activities of the proteasome include include chymotrypsin-like activity (cleavage after hydrophobic side chains), postglutamyl peptidase activity (cleavage after acidic side chains), trypsin-like activity (cleavage after basic side chains) and tripeptidyl peptidase II (TPPII), which is believed to participate in the degradation of extra-lysosomal polypeptides and may substitute for some metabolic functions of the proteasome, particularly in the absence of normal proteasome function.

Compounds which may be used are those that are capable of blocking proteasome function without affecting the normal biological processes in the cell.

Examples of suitable ubiquitin-proteasome pathway protein inhibitors include tripeptide aldehyde compounds, which are known to be reversible inhibitors of chymotrypsin-like, postglutamyl, and trypsin-like activities of the proteasome. Another class of compounds, vinyl sulfones, act as suicide substrates for the active site nucleophiles, and may be used as described here for the treatment or alleviation of dengue. Lactacystin, a third group compound, is a covalent inhibitor of the chymotrypsin-like and trypsin-like activities of the proteasome. Its action is thought to be due to the action of its β-lactone form that is produced upon incubation in the aqueous medium. Some of these inhibitors also have a significant inhibitory effect on the activity of tripeptidyl peptidase II. Lactacystin group compounds may also be employed in the treatment or alleviation of dengue.

| | Calbiochem** | |
|---|---|---|
| Proteasome | Catalogue | |
| Inhibitor | Number | Description |
| Aclacinomycin | 112270 | An anthracycline antitumor agent that inhibits |
| A, Streptomyces | | the degradation of ubiquitinated proteins by |
| galilaeus | | blocking the chymotrypsin-like activity of the |
| | | 20S proteasome. Also inhibits DNA |
| | | topoisomerase I and II. |
| Calpain Inhibitor | 208719 | Ubiquitin-dependent inhibitor of proteolysis of |
| I (MG-101) | | I\| B-αand I\| B-β by the ubiquitinproteasome |
| | | complex. Also inhibits calpain I, calpain II, |
| | | cathepsin B, and cathepsin L (Ki = 150-500 nM). |
| | | Sequence: Ac-Leu-Leu-Norleucinal |
| Calpain Inhibitor | 208721 | Inhibits calpains I and II, cathepsin B, and |
| II | | cathepsin L (Ki = 120-600 nM) but does not |
| | | inhibit proteasome activity. Serves as a negative |
| | | control for Calpain inhibitor I (Cat. No. 208719). |
| | | Sequence: Ac-Leu-Leu-Methional |
| clasto- | 426102 | Cell-permeable, active component of Lactacystin |
| Lactacystin β- | | (Cat. No. 426100). Irreversible and highly |
| Lactone | | specific inhibitor of the 20S proteasome. About |
| | | 10-fold more sensitive than its precursor, |
| | | lactacystin, in vitro. |
| Lactacystin | 426100 | Cell-permeable, irreversible, specific inhibitor of |
| | | the trypsin-like and chymotrypsin-like activities |
| | | of the 20S proteasome (IC50 ~1 μM). Blocks |
| | | proteasome activity by targeting the catalytic β- |
| | | subunit of the proteasome by covalently |
| | | attaching to the N-terminal Thr of subunit X |
| | | (MB1). Induces apoptosis in human monoblastic |
| | | U937 cells. |
| MG-115 | 474780 | Potent reversible proteasome inhibitor (Ki = 21 nM |
| | | and 35 nM for 20S and 26S proteasome, |
| | | respectively). Blocks the assembly of class I |
| | | molecules by inhibiting generation of peptides |
| | | presented on MHC class I molecules. Sequence: |
| | | Z-Leu-Leu-Norvalinal |
| MG-132 | 474790 | Cell-permeable, potent, reversible inhibitor. |
| | | Reduces the degradation of ubiquitinconjugated |
| | | proteins by the 26S complex without affecting |
| | | its ATPase or isopeptidase activities. Effective at |
| | | micromolar concentrations. Sequence: Z-Leu- |
| | | Leu-Leucinal |
| NLVS | 482240 | Cell-permeable, irreversible inhibitor of the |
| | | trypsin-like, chymotrypsin-like, and peptidyl- |
| | | glutamyl peptidase activities of proteasomes. |
| | | Acts by covalently modifying the NH2-terminal |
| | | Thr of the catalytically active β subunit. |
| NP-LLL-VS | 492025 | An intermediate that can be used to prepare |
| | | 125I-radiolabeled NLVS (Cat. No. 482240) for |
| | | proteasome inhibition studies. |
| Proteasome | 539160 | Cell-permeable inhibitor of the chymotrypsin- |
| Inhibitor I | | like activity of the 20S proteasome. Causes |
| | | accumulation of ubiquitinated proteins. Blocks |
| | | activation of NF-κB in macrophages. ID50 = 250 nM |
| | | in vitro; 15 μM in cells. Sequence: Z-Ile- |
| | | Glu(OtBu)-Ala-Leucinal |
| Proteasome | 539162 | Potent, cell-permeable proteasome inhibitor. |
| Inhibitor II | | Inhibits the chymotrypsin-like activity (Ki = 460 nM), |
| | | but not the peptidylglutamyl-hydrolyzing |
| | | activity. Also blocks the decay of I\| B-αand I\| B- |
| | | βproteins in WEHI 231 cells. Sequence: Z-Leu- |
| | | Leu-Phenylalaninal |
| Ubiquitin | 662056 | Stabilizes endogenous or in vitro-synthesized |
| Aldehyde | | ubiquitin-protein conjugates. Potent, specific |
| | | inhibitor of ubiquitin hydrolases and ubiquitin- |
| | | protein isopeptidases involved in intracellular |
| | | modification or turnover. |

**Calbiochem, San Diego, USA

Alternatively, or in addition, the proteasome inhibitor may comprise an antibody against a component of the proteasome, for example any of the following (Calbiochem catalogue numbers in brackets): Anti-20S Proteasome, α-Subunit, Methanosarcina thermophila (Rabbit) (539153); Anti-20S Proteasome, β-Subunit, Methanosarcina thermophila (Rabbit) (539156); Anti-Ubiquitin, Bovine Erythrocyte (Mouse) (662097); Anti-Ubiquitin-Activating Enzyme E1A, N-Terminal, Human (Rabbit) (662102); Anti-Ubiquitin-Activating Enzyme MB, N-Terminal, Human (Rabbit) (662104); Anti-Ubiquitin-Activating Enzyme E1A/E1B, C-Terminal, Human (Rabbit) (662106).

In some embodiments, the ubiquitin-proteasome pathway protein inhibitor comprises MG-132 or ALLN.

MG-132 (Carbobenzoxy-L-Leucyl-L-Leucyl-L-Leucinal)

MG-132 is a potent, reversible, and cell-permeable proteasome inhibitor (Ki=4 nM). MG-132 reduces the degradation of ubiquitin-conjugated proteins in mammalian cells and permeable strains of yeast by the 26S complex without affecting its ATPase or isopeptidase activities. It has a CAS registry number CAS133407-82-6 and the formula Carbobenzoxy-L-leucyl-L-leucyl-L-leucinal

MG-132 is also known as Z-LLL-CHO. It is a white solid. MG-132 activates c-Jun N-terminal kinase (JNK1), which initiates apoptosis. It inhibits NF-kB activation (IC50=3 μM) and prevents b-secretase cleavage.

MG-132 and its effects is described in Steinhilb, M. L., et al. 2001. J. Biol. Chem. 276, 4476. Merlin, A. B., et al. 1998. J. Biol. Chem. 273, 6373. Adams, J., and Stein, R. 1996. Ann. Rep. Med. Chem. 31, 279. Klafki, H. W., et al. 1996. J. Biol. Chem. 271, 2865. Lee, D. H., and Goldberg, A. L., 1996. J. Biol. Chem. 271, 27280. Wiertz, E. J., et al. 1996. Cell 84, 769. Jensen, T. J., et al. 1995. Cell 83, 129. Read, M. A., et al. 1995. Immunity 2, 493. Rock, K. L., et al. 1994. Cell 78, 761.

ALLN (N-Acetyl-Leu-Leu-Nle-CHO)

ALLN is an inhibitor of the proteolysis of IkB-a and IkB-b by the ubiquitin-proteasome complex. It has a CAS registry number CAS110044-82-1 and the formula N-Acetyl-Leu-Leu-Nle-CHO.

ALLN is also known as Calpain Inhibitor I, LLNL and MG 101. It is a white to off-white solid and is a cell-permeable inhibitor of calpain I (Ki=190 nM), calpain II (Ki=220 nM), cathepsin B (Ki=150 nM), and cathepsin L (Ki=500 pM). ALLN inhibits neutral cysteine proteases and the proteasome (Ki=6 μM), and modulates the processing of the b-amyloid precursor protein (bAPP) to b-amyloid (Ab). It protects against neuronal damage caused by hypoxia and ischemia and inhibits apoptosis in thymocytes and metamyelocytes. ALLN also inhibits reovirus-induced apoptosis in L929 cells.

ALLN inhibits cell cycle progression at G1/S and metaphase/anaphase in CHO cells by inhibiting cyclin B degradation, and also prevents nitric oxide production by activated macrophages by interfering with transcription of the inducible nitric oxide synthase gene.

ALLN and its effects is described in Debiasi, R. L., et al. 1999. J. Virol. 73, 695. Zhang, L., et al. 1999. J. Biol. Chem. 274, 8966. Milligan, S. A., et al. 1996. Arch. Biochem. Biophys. 335, 388. Griscavage, J. M., et al. 1995. Biochem. Biophys. Res. Commun. 215, 721. Squier, M. K., et al. 1994. J. Cell Physiol. 159, 229. Rami, J., and Kreiglstein, J. 1993. Brain Res. 609, 67. Sherwood, S. W., et al. 1993. Proc. Natl. Acad. Sci. USA 90, 3353. Vinitsky, A., et al. 1992. Biochemistry 31, 9421. Sasaki, T., et al. 1990. J. Enzyme Inhib. 3, 195.

### Interferon-Related Protein

By the term “interferon-related protein”, we mean any of the genes whose expression, activity and/or function is mediated or modulated by interferon. The interferon-related protein may be a interferon-mediated protein.

In some embodiments, the interferon-related protein or interferon-mediated protein is a protein set out in Table D2 below.

TABLE D2
Interferon-Related Proteins

| | GenBank |
|---|---|
| Gene | Accession Number |
| AIM2 | NM_004833 |
| ATF3 | NM_004024 |
| G1P2 | NM_005101 |
| G1P3 | NM_002038 |
| GBP1 | NM_002053 |
| GBP2 | NM_004120 |
| GBP4 | NM_052941 |
| GBP5 | NM_052942 |
| IER3 | NM_003897 |
| IFI16 | NM_005531 |
| IFI44 | NM_006417 |
| IFIH1 | AL080107 |
| IFIT1 | NM_001548 |
| IFIT2 | AF026944 |
| IFIT3 | AF026943 |
| IFIT5 | NM_012420 |
| IFNA1 | NM_024013 |
| IFNB1 | NM_002176 |
| IFNG | NM_000619 |
| IFRG28 | AJ251832 |
| IRF1 | NM_002198 |
| IRF7 | NM_004030 |
| IRF9 | NM_006084 |
| ISG15 | NM_005101 |
| ISGF3G | NM_006084 |
| MDA5 | AF095844 |
| MKP-1 | NM_004417, AJ227912 |
| MxA | NM_002462 |
| NMI | NM_004688 |
| OAS1 | NM_016816 |
| OAS2 | NM_002535 |
| OAS3 | NM_006187 |
| OASL | AF063611 |
| SLAMF7 | NM_021181 |
| SOCS1 | NM_003745 |
| SP110 | NM_004510 |
| STAT1 | AK022231, NM_007315 |
| STAT2 | NM_005419 |
| Viperin | AF026941, AF026942 |

For example, the interferon-related protein could comprise interferon alpha.

NK-⊃B-Mediated Cytokine/Chemokine Response Protein

By the term “NF-κB-mediated cytokine/chemokine response protein”, we mean any of the genes whose expression, activity and/or function is mediated or modulated by NF-κB. Specifically, the NF-κB-mediated cytokine/chemokine response protein comprises a cytokine or a chemokine . . . .

The NF-κB-mediated cytokine/chemokine response protein may comprise a mediator protein.

In some embodiments, the NF-κB-mediated cytokine/chemokine response protein is a protein set out in Table D3 below.

TABLE D3
NF-κB-mediated cytokine/chemokine response protein

| | GenBank |
|---|---|
| Gene | Accession Number |
| B2M | NM_004048 |
| CARD15 | NM_022162 |
| CARD4 | NM_006092 |
| CCL2 | NM_002982 |
| CCL4 | NM_002984 |
| CCL5 | NM_002985 |
| CCR1 | NM_001295 |
| CCR5 | NM_000579 |
| CCR7 | NM_001838 |
| CCRL2 | NM_003965 |
| CD14 | NM_000591 |
| CD1A | NM_001763 |
| CD2 | NM_001767 |
| CD22 | NM_001771 |
| CD276 | NM_025240 |
| CD47 | NM_001777 |
| CD59 | NM_000611 |
| CD97 | NM_001784 |
| COX2 | NM_000963 |
| CXCL16 | NM_022059 |
| IL10 | NM_000572 |
| IL10RB | NM_000628 |
| IL11b | NM_000881 |
| IL13RA1 | NM_001560 |
| IL16 | NM_004513 |
| IL18 | NM_001562 |
| IL18RAP | NM_003853 |
| IL1RN | NM_173842 |
| IL2 | NM_000586 |
| IL4R | NM_000418 |
| IL6 | NM_000600 |
| IL8 | M17017 |
| IL8RA | NM_000634 |
| IL8RB | NM_001557 |
| INOS | NM_000625 |
| IP-10 | NM_001565 |
| I-TAC | NM_005409 |
| NFKBIA | NM_020529 |
| NFKBIB | NM_002503 |
| PAI1 | NM_000602 |
| PBEF1 | NM_182790 |
| PF4 | NM_002619 |
| RANTES | NM_002985 |
| RIG-I | NM_014314 |
| TNF | NM_000594 |
| TNFAIP2 | NM_006291 |
| TNFAIP3 | NM_006290 |
| TNFAIP8 | NM_014350 |
| TNFRSF1A | NM_001065 |
| TNFRSF1B | NM_001066 |
| TNFRSF25, | NM_148970 |
| TNFRSF7 | NM_001242 |
| TNFSF10 | NM_003810 |
| VEGF | NM_003376 |
| B2M | NM_004048 |
| CARD15 | NM_022162 |
| CARD4 | NM_006092 |
| CCL2 | NM_002982 |
| CCL4 | NM_002984 |
| CCL5 | NM_002985 |
| CCR1 | NM_001295 |

### Screening Assays

The ubiquitin-proteasome pathway protein, interferon-related protein or NF-κB-mediated cytokine/chemokine response protein, including homologues, variants, and derivatives, whether natural or recombinant, may be employed in a screening process for compounds which bind the protein and which activate (agonists) or inhibit activation of (antagonists) of the protein. Such agonists and antagonists may be used in the treatment, prevention or alleviation of dengue.

Thus, the ubiquitin-proteasome pathway protein, interferon-related protein or NF-κB-mediated cytokine/chemokine response protein, as the case may be, may also be used to assess the binding of small molecule substrates and ligands in, for example, cells, cell-free preparations, chemical libraries, and natural product mixtures. These substrates and ligands may be natural substrates and ligands or may he structural or functional mimetics. See Coligan et al., Current Protocols in Immunology 1(2):Chapter 5 (1991).

As described herein, inhibitors of ubiquitin-proteasome pathway proteins, interferon-related proteins or NF-κB-mediated cytokine/chemokine response proteins may be used to target dengue viral function, and for the treatment or alleviation of symptoms of dengue fever.

Accordingly, it is desirous to find compounds and drugs which stimulate ubiquitin-proteasome pathway proteins, interferon-related proteins or NF-κB-mediated cytokine/chemokine response proteins on the one hand and which can inhibit the function of ubiquitin-proteasome pathway proteins, interferon-related proteins or NF-κB-mediated cytokine/chemokine response proteins on the other hand. In general, agonists and antagonists are employed for therapeutic and prophylactic purposes for dengue infection.

An agonist may activate the ubiquitin-proteasome pathway protein, interferon-related protein or NF-κB-mediated cytokine/chemokine response protein (as the case may be) to any degree. Similarly, an antagonist may deactivate, or inhibit the activation of, the ubiquitin-proteasome pathway protein, interferon-related protein or NF-κB-mediated cytokine/chemokine response protein to any degree. The protein may therefore be deactivated partially to any degree to its inherent, basal or background level of activity by an antagonist (partial antagonist) or fully to such a level (antagonist or full antagonist). The antagonist may deactivate the protein even further, for example to zero activity (inverse agonist). The term “antagonist” therefore specifically includes both full antagonists, partial antagonists and inverse agonists.

Also included within the terms “agonist” and “antagonist” are those molecules which modulate the expression of a ubiquitin-proteasome pathway protein, a interferon-related protein or an NF-κB-mediated cytokine/chemokine response protein, as the case may be, at the transcriptional level and/the translational level, as well as those which modulate its activity.

Rational design of candidate compounds likely to be able to interact with a ubiquitin-proteasome pathway protein, interferon-related, protein or NF-κB-mediated cytokine/chemokine response protein may be based upon structural studies of the molecular shapes of a polypeptide. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

An alternative to rational design uses a screening procedure which involves in general allowing a ubiquitin-proteasome pathway protein, interferon-related protein or NF-κB-mediated cytokine/chemokine response protein to contact a candidate modulator and detecting an effect thereof. In general, such a method comprises producing appropriate cells which express the relevant protein or polypeptide on the surface thereof, optionally together with a partner protein, and contacting the protein or the cell or both with a candidate modulator, and detecting a change in the intracellular level of a relevant molecule.

A candidate compound may be tested for the ability to inhibit an activity of a ubiquitin-proteasome pathway protein, interferon-related protein or NF-κB-mediated cytokine/chemokine response protein. For example, a candidate molecule may be assayed by assaying its effect on proteasome activity. Such assays may make use of specific substrates, for example, the substrates provided in Proteasome Inhibitor Set (Calbiochem Cat. No. 539164), which contains 1 mg Proteasome Inhibitor I (Cat. No. 539160), 1 mg MG-132 (Cat. No. 474790), and 200 μg Lactacystin (Cat. No. 426100). A kit for assaying activity of the 26S proteasome is available as Cat. No. 539159 from Calbiochem (San Diego, USA).

Antibody Based Assay

We provide preliminary data that IP-10 and I-TAC serve as biomarkers for the viremic phase in early dengue patient samples compared to serum samples from other febrile patients. The two proteins can be detected readily using sensitive antibody based tests which together with a host antigen such as NS1 (Non-Structural protein 1) can form the basis of a diagnostic assay that can be readily adapted into a test that can be used in the field.

Molecules whose concentrations are affected by activity of ubiquitin-proteasome pathway proteins, interferon-related proteins or NF-κB-mediated cytokine/chemokine response proteins, and which may be used as markers for detecting protein activity, are known in the art. These are referred to for convenience as “protein sensitive markers”, and these may be detected as a means of detecting activity of the relevant protein.

Cells which may be used for the screen may be of various types. Such cells include cells from animals, yeast, Drosophila or E. coli. Cells expressing the ubiquitin-proteasome pathway protein, interferon-related protein or NF-κB-mediated cytokine/chemokine response protein are then contacted with a test compound to observe binding, or stimulation or inhibition of a functional response.

Instead of testing each candidate compound individually with the ubiquitin-proteasome pathway protein, interferon-related protein or NF-κB-mediated cytokine/chemokine response protein, a library or bank of candidate molecules may advantageously be produced and screened.

Where the candidate compounds are proteins, in particular antibodies or peptides, libraries of candidate compounds may be screened using phage display techniques. Phage display is a protocol of molecular screening which utilises recombinant bacteriophage. The technology involves transforming bacteriophage with a gene that encodes one compound from the library of candidate compounds, such that each phage or phagemid expresses a particular candidate compound. The transformed bacteriophage (which may be tethered to a solid support) expresses the appropriate candidate compound and displays it on their phage coat. Specific candidate compounds which are capable of binding to a ubiquitin-proteasome pathway protein, interferon-related protein or NF-κB-mediated cytokine/chemokine response protein, polypeptide or peptide are enriched by selection strategies based on affinity interaction. The successful candidate agents are then characterised. Phage display has advantages over standard affinity screening technologies. The phage surface displays the candidate agent in a three dimensional configuration, more closely resembling its naturally occurring conformation. This allows for more specific and higher affinity binding for screening purposes.

Another method of screening a library of compounds utilises eukaryotic or prokaryotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al. (1990) Proc. Nat'l Acad. Sci. USA 87;4007-4011, which describe sensitive methods to detect cellular responses. Competitive assays are particularly useful, where the cells expressing the library of compounds are contacted or incubated with a labelled antibody known to bind to a ubiquitin-proteasome pathway protein, interferon-related protein or NF-κB-mediated cytokine/chemokine response protein, such as ¹²⁵I-antibody, and a test sample such as a candidate compound whose binding affinity to the binding composition is being measured. The bound and free labelled binding partners for the polypeptide are then separated to assess the degree of binding. The amount of test sample bound is inversely proportional to the amount of labelled antibody binding to the polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to a ubiquitin-proteasome pathway protein, interferon-related protein or NF-κB-mediated cytokine/chemokine response protein, and is described in detail in International Patent application no. WO 84/03564 (Commonwealth Serum Labs.), published on Sep. 13 1984. First, large numbers of different small peptide test compounds are synthesized on a solid substrate, e.g., plastic pins or some other appropriate surface; see Fodor et al. (1991). Then all the pins are reacted with solubilized polypeptide and washed. The next step involves detecting bound polypeptide. Compounds which interact specifically with the polypeptide will thus be identified.

The assays may simply test binding of a candidate compound wherein adherence to the cells bearing the relevant protein is detected by means of a label directly or indirectly associated with the candidate compound or in an assay involving competition with a labeled competitor. Further, these assays may test whether the candidate compound results in a signal generated by activation of the protein, using detection systems appropriate to the cells bearing the protein. Inhibitors of activation are generally assayed in the presence of a known agonist and the effect on activation by the agonist by the presence of the candidate compound is observed.

Further, the assays may simply comprise the steps of mixing a candidate compound with a solution containing a ubiquitin-proteasome pathway protein, interferon-related protein or NF-κB-mediated cytokine/chemokine response protein to form a mixture, measuring ubiquitin-proteasome pathway protein, interferon-related protein or NF-κB-mediated cytokine/chemokine response protein (as the case may be) activity in the mixture, and comparing the protein activity of the mixture to a standard.

The ubiquitin-proteasome pathway protein, interferon-related protein or NF-κB-mediated cytokine/chemokine response protein cDNA, protein and antibodies to the protein may also be used to configure assays for detecting the effect of added compounds on the production of the relevant mRNA and protein in cells. For example, an ELISA may be constructed for measuring secreted or cell associated levels of ubiquitin-proteasome pathway protein, interferon-related protein or NF-κB-mediated cytokine/chemokine response protein using monoclonal and polyclonal antibodies by standard methods known in the art, and this can be used to discover agents which may inhibit or enhance the production of ubiquitin-proteasome pathway protein, interferon-related protein or NF-κB-mediated cytokine/chemokine response protein (also called antagonist or agonist, respectively) from suitably manipulated cells or tissues. Standard methods for conducting screening assays are well understood in the art.

The screening assays may be conducted in vitro, as described above, or in vivo. In vivo assays may in particular be conducted using cells or suitable animal models for dengue.

Animal models for dengue include mice and monkeys. Such animal models are described in detail in Dengue Digest, volume 2, number 4, December 2005 (MICA (P) 193/06/2005; Novartis Institute for Tropical Diseases, Singapore); this document is available at http://www.nitd.novartis.com/includes/teasers/teaser_attaches/dengue_digest/DengueDigest_v2n4.pdf. A mouse model for dengue fever is described in Schul W, Liu W, Xu H Y, Flamand M, Vasudevan S G. (2007), A dengue fever viremia model in mice shows reduction in viral replication and suppression of the inflammatory response after treatment with antiviral drugs. J Infect Dis. 2007 Mar. 1;195(5):665-74. Epub 2007 Jan. 23.

Any of the suitable animal models described in these documents may be used for the screening assays described here.

An example of such an assay employs administering a candidate molecule to an animal suffering from dengue, and detecting a change in a parameter indicative of dengue infection or progression, such as viral load, viral replication, any symptom of dengue etc. Candidate molecules which alleviate or reduce dengue symptoms, including viral replication, may be useful as drugs for the prevention, alleviation or treatment of dengue.

Examples of potential ubiquitin-proteasome pathway protein, interferon-related protein or NF-κB-mediated cytokine/chemokine response protein antagonists include small molecules, antibodies or, in some cases, nucleotides and their analogues, including purines and purine analogues, oligonucleotides or proteins.

We therefore also provide a compound capable of binding specifically to a ubiquitin-proteasome pathway protein, interferon-related protein or NF-κB-mediated cytokine/chemokine response protein and/or peptide.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. The compound may be an antibody.

The materials necessary for such screening to be conducted may be packaged into a screening kit. Such a screening kit is useful for identifying agonists and antagonists, for ubiquitin-proteasome pathway proteins, interferon-related proteins or NF-κB-mediated cytokine/chemokine response proteins or compounds which decrease or enhance the production of such polypeptides. The screening kit comprises: (a) a ubiquitin-proteasome pathway protein, interferon-related protein or NF-κB-mediated cytokine/chemokine response protein; (b) a recombinant cell expressing a ubiquitin-proteasome pathway protein, interferon-related protein or NF-κB-mediated cytokine/chemokine response polypeptide; (c) a cell membrane expressing a ubiquitin-proteasome pathway protein, interferon-related protein or NF-κB-mediated cytokine/chemokine response protein; or (d) antibody to a ubiquitin-proteasome pathway protein, interferon-related protein or NF-κB-mediated cytokine/chemokine response protein. The screening kit may optionally comprise instructions for use.

### Variants and Functional Equivalents of ALLN and MG-132

We provide for the use of compounds which are variants of, or similar in structure or functionally equivalent to MG-132 and/or ALLN for the purposes described in this document. These are described in detail below.

Chemical Derivative of ALLN and MG-132

The term “derivative” or “derivatised” as used herein includes chemical modification of a compound. Illustrative of such chemical modifications would be replacement of hydrogen by a halo group, an alkyl group, an acyl group or an amino group.

Chemical Modification of ALLN and MG-132

In one embodiment, the ALLN and/or MG-132 or variant thereof may be a chemically modified compound.

The chemical modification of a compound may either enhance or reduce hydrogen bonding interaction, charge interaction, hydrophobic interaction, Van Der Waals interaction or dipole interaction between the compound and the target.

Substituted Variants of ALLN and MG-132

We provide for the use of substituted variants of ALLN and MG-132. For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

Pharmaceutically Acceptable Salts of ALLN and MG-132

The the ALLN and/or MG-132 or variant thereof may be in the form of—and/or may be administered as—a pharmaceutically acceptable salt—such as an acid addition salt or a base salt—or a solvate thereof, including a hydrate thereof. For a review on suitable salts see Berge et al. J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

Polymorphic Form(s)/Asymmetric Carbon(s) of ALLN and MG-132

The ALLN and/or MG-132 or variant thereof may exist in polymorphic form.

Such a compound may contain one or more asymmetric carbon atoms and therefore exists in two or more stereoisomeric forms. Where a compound contains an alkenyl or alkenylene group, cis (E) and trans (Z) isomerism may also occur. Individual stereoisomers of the ALLN and/or MG-132 or variant thereof and, where appropriate, the individual tautomeric forms thereof, together with mixtures thereof, are also included within this disclosure.

Separation of diastereoisomers or cis and trans isomers may be achieved by conventional techniques, e.g. by fractional crystallisation, chromatography or H.P.L.C. of a stereoisomeric mixture of the compound or a suitable salt or derivative thereof An individual enantiomer of the compound may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Isotopic Variations of ALLN and MG-132

The present disclosure also includes all suitable isotopic variations of the compounds or a pharmaceutically acceptable salt thereof.

An isotopic variation of the ALLN and/or MG-132 or variant thereof or a pharmaceutically acceptable salt thereof is defined as one in which at least one atom is replaced by an atom having the same atomic number but an atomic mass different from the atomic mass usually found in nature. Examples of isotopes that can be incorporated into the compounds and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the compounds and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds and pharmaceutically acceptable salts thereof can generally be prepared by conventional procedures using appropriate isotopic variations of suitable reagents.

Prodrugs of ALLN and MG-132

It will be appreciated by those skilled in the art that the compounds may be derived from a prodrug. Examples of prodrugs include entities that have certain protected group(s) and which may not possess pharmacological activity as such, but may, in certain instances, be administered (such as orally or parenterally) and thereafter metabolised in the body to form the compound which are pharmacologically active.

Pro-Moieties of ALLN and MG-132

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the compounds. Such prodrugs are also included within this disclosure.

Salts of ALLN and MG-132

The ALLN and/or MG-132 or variant thereof can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, sulfate, 3-phenylpropionate, picrate, pivalate, propionate, succinate, tartrate, thiocyanate, p-toluenesulfonate and undecanoate. Also, the basic nitrogen-containing groups can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oilsoluble or dispersible products are thereby obtained.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. Basic addition salts can be prepared in situ during the final isolation and purification of the ALLN and/or MG-132 or variant thereof, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

HUMAN (IN VIVO) ASSOCIATED GENES

| | Genbank | | |
|---|---|---|---|
| Gene | Accession | | |
| Symbol | Number | Synonyms | Description |
| USP18 | NM_017414.2 | ISG43; UBP43 | ubiquitin specific protease 18 (USP18), |
| | | | mRNA. |
| UBE2L6 | NM_004223.3 | RIG-B; UBCH8; | ubiquitin-conjugating enzyme E2L 6 |
| | | MGC40331 | (UBE2L6), transcript variant 1, mRNA. |
| UBE2J2 | NM_194457.1 | NCUBE2; PRO2121 | ubiquitin-conjugating enzyme E2, J2 |
| | | | (UBC6 homolog, yeast) (UBE2J2), |
| | | | transcript variant 4, mRNA. |
| UBE2L6 | NM_004223.3 | RIG-B; UBCH8; | ubiquitin-conjugating enzyme E2L 6 |
| | | MGC40331 | (UBE2L6), transcript variant 1, mRNA. |
| ITCH | NM_031483.3 | AIF4; AIP4; NAPP1 | itchy homolog E3 ubiquitin protein |
| | | | ligase (mouse) (ITCH), mRNA. |
| UBE1L | NM_003335.2 | D8; UBE2; MGC12713 | ubiquitin-activating enzyme E1-like |
| | | | (UBE1L), mRNA. |
| UBAP1 | NM_016525.3 | UAP; UBAP; NAG20; | ubiquitin associated protein 1 (UBAP1), |
| | | MGC8710 | mRNA. |
| UBE2R2 | NM_017811.2 | UBC3B; CDC34B; | ubiquitin-conjugating enzyme E2R 2 |
| | | FLJ20419 | (UBE2R2), mRNA. |
| USP32 | NM_032582.3 | USP10; NY-REN-60 | ubiquitin specific protease 32 (USP32), |
| | | | mRNA. |
| UBE2V1 | NM_022442.3 | CIR1; UEV1; CROC1; | ubiquitin-conjugating enzyme E2 |
| | | UEV-1 | variant 1 (UBE2V1), transcript variant |
| | | | 3, mRNA. |
| UBE2I | NM_194260.1 | P18; UBC9; C358B7.1 | ubiquitin-conjugating enzyme E2I |
| | | | (UBC9 homolog, yeast) (UBE2I), |
| | | | transcript variant 3, mRNA. |
| USP39 | NM_006590.1 | SAD1; CGI-21 | ubiquitin specific protease 39 (USP39), |
| | | | mRNA. |
| UBE2A | NM_181762.1 | UBC2; HHR6A; | ubiquitin-conjugating enzyme E2A |
| | | RAD6A | (RAD6 homolog) (UBE2A), transcript |
| | | | variant 2, mRNA. |
| UBE2E3 | NM_182678.1 | UBCH9; UbcM2 | ubiquitin-conjugating enzyme E2E 3 |
| | | | (UBC4/5 homolog, yeast) (UBE2E3), |
| | | | transcript variant 2, mRNA. |
| USP49 | NM_004275.2 | TRFP; PRO0213; | ubiquitin specific protease 49 (USP49), |
| | | DKFZp586D2223 | mRNA. |
| UBASH3A | NM_018961.1 | | ubiquitin associated and SH3 domain |
| | | | containing, A (UBASH3A), mRNA. |
| UBE4A | NM_004788.2 | E4; UFD2; KIAA0126 | ubiquitination factor E4A (UFD2 |
| | | | homolog, yeast) (UBE4A), mRNA. |
| USP47 | NM_017944.2 | FLJ20727 | ubiquitin specific protease 47 (USP47), |
| | | | mRNA. |
| USP38 | NM_032557.4 | HP43.8KD; KIAA1891 | ubiquitin specific protease 38 (USP38), |
| | | | mRNA. |
| UBE1C | NM_198197.1 | UBA3; hUba3; | ubiquitin-activating enzyme E1C |
| | | MGC22384; | (UBA3 homolog, yeast) (UBE1C), |
| | | | transcript variant 3, mRNA. |
| UBL3 | NM_007106.2 | HCG-1; PNSC1; | ubiquitin-like 3 (UBL3), mRNA. |
| | | DKFZP434K151 | |
| KIAA0010 | NM_014671.1 | | ubiquitin-protein isopeptide ligase (E3) |
| | | | (KIAA0010), mRNA. |
| USP34 | NM_014709.2 | KIAA0570; KIAA0729 | ubiquitin specific protease 34 (USP34), |
| | | | mRNA. |
| UCHL3 | NM_006002.3 | | ubiquitin carboxyl-terminal esterase L3 |
| | | | (ubiquitin thiolesterase) (UCHL3), |
| | | | mRNA. |
| USP52 | NM_014871.2 | PAN2; KIAA0710 | ubiquitin specific protease 52 (USP52), |
| | | | mRNA. |
| UBE4B | NM_006048.2 | E4; 686; UFD2; | ubiquitination factor E4B (UFD2 |
| | | HDNB1; KIAA0684 | homolog, yeast) (UBE4B), mRNA. |
| HERPUD1 | NM_014685.1 | SUP; HERP; Mif1; | homocysteine-inducible, endoplasmic |
| | | KIAA0025 | reticulum stress-inducible, ubiquitin- |
| | | | like domain member 1 (HERPUD1), |
| | | | mRNA. |
| UBAP2 | NM_020867.1 | FLJ22435; KIAA1491; | ubiquitin associated protein 2 (UBAP2), |
| | | bA176F3.5 | transcript variant 2, mRNA. |
| USP7 | NM_003470.1 | TEF1; HAUSP | ubiquitin specific protease 7 (herpes |
| | | | virus-associated) (USP7), mRNA. |
| HSPC150 | NM_014176.1 | | HSPC150 protein similar to ubiquitin- |
| | | | conjugating enzyme (HSPC150), |
| | | | mRNA. |
| USP1 | NM_003368.3 | | ubiquitin specific protease 1 (USP1), |
| | | | mRNA. |
| USP16 | NM_006447.1 | Ubp-M | ubiquitin specific protease 16 (USP16), |
| | | | mRNA. |
| UBE3A | NM_130838.1 | AS; ANCR E6-AP; | ubiquitin protein ligase E3A (human |
| | | EPVE6AP | papilloma virus E6-associated protein, |
| | | | Angelman syndrome) (UBE3A), |
| | | | transcript variant 1, mRNA. |
| UBE2N | NM_003348.3 | UBC13; MGC8489; | ubiquitin-conjugating enzyme E2N |
| | | UbcH-ben | (UBG13 homolog, yeast) (UBE2N), |
| | | | mRNA. |
| UCHL5 | NM_015984.1 | UCH37; CGI-70 | ubiquitin carboxyl-terminal hydrolase |
| | | | L5 (UCHL5), mRNA. |
| UFD1L | NM_005659.3 | | ubiquitin fusion degradation 1-like |
| | | | (UFD1L), mRNA. |
| UBL5 | NM_024292.2 | HUB1 | ubiquitin-like 5 (UBL5), mRNA. |
| UBE2G1 | NM_003342.3 | UBC7; E217K; | ubiquitin-conjugating enzyme E2G 1 |
| | | UBE2G | (UBC7 homolog, C. elegans) |
| | | | (UBE2G1), transcript variant 1, mRNA. |
| UBE2V2 | NM_003350.2 | MMS2; UEV2; | ubiquitin-conjugating enzyme E2 |
| | | EDPF1; UEV-2; | variant 2 (UBE2V2), mRNA. |
| UBE2J1 | NM_016336.2 | Ubc6p; CGI-76; | ubiquitin-conjugating enzyme E2, J1 |
| | | NCUBE1; HSPC153; | (UBC6 homolog, yeast) (UBE2J1), |
| | | | mRNA. |
| USP28 | NM_020886.2 | KIAA1515 | ubiquitin specific protease 28 (USP28), |
| | | | mRNA. |
| UBE2J1 | NM_016021.2 | NCUBE1; HSPC153; | ubiquitin-conjugating enzyme E2, J1 |
| | | HSPC205; | (UBC6 homolog, yeast) (UBE2J1), |
| | | | mRNA. |
| UBE2G2 | NM_003343.4 | UBC7 | ubiquitin-conjugating enzyme E2G 2 |
| | | | (UBC7 homolog, yeast) (UBE2G2), |
| | | | transcript variant 1, mRNA. |
| UHRF1 | NM_013282.2 | Np95; ICBP90; | ubiquitin-like, containing PHD and |
| | | RNF106; FLJ21925 | RING finger domains, 1 (UHRF1), |
| | | | mRNA. |
| UBE2C | NM_181803.1 | UBCH10; dJ447F3.2 | ubiquitin-conjugating enzyme E2C |
| | | | (UBE2C), transcript variant 6, mRNA. |

### Pharmaceutical Compositions

As disclosed herein, inhibitors, agonists or antagonists of (a) a ubiquitin-proteasome pathway protein; (b) a interferon-related protein; or (c) an NF-κB-mediated cytokine/chemokine response protein may be used to treat or prevent dengue.

Inhibitors, agonists or antagonists of ubiquitin-proteasome pathway proteins, interferon-related proteins or NF-κB-mediated cytokine/chemokine response proteins can be administered in a variety of ways including enteral, parenteral and topical routes of administration. For example, suitable modes of administration include oral, subcutaneous, transdermal, transmucosal, iontophoretic, intravenous, intramuscular, intraperitoneal, intranasal, subdural, rectal, and the like.

In accordance with other embodiments, there is provided a composition comprising an ubiquitin-proteasome pathway protein inhibitor, which in some embodiments comprises MG-132 or ALLN, together with a pharmaceutically acceptable carrier or excipient for the treatment or prevention of dengue.

Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-p-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), incorporated herein by reference.

Pharmaceutical compositions containing inhibitors, agonists or antagonists of ubiquitin-proteasome pathway proteins, interferon-related proteins or NF-κB-mediated cytokine/chemokine response proteins such as MG-132 or ALLN may be in any form suitable for the intended method of administration, including, for example, a solution, a suspension, or an emulsion. Liquid carriers are typically used in preparing solutions, suspensions, and emulsions. Liquid carriers contemplated for use in the practice include, for example, water, saline, pharmaceutically acceptable organic solvent (s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols.

Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions may also be in the form of microparticles, microcapsules, liposomal encapsulates, and the like, as well as combinations of any two or more thereof.

The inhibitors, agonists or antagonists of ubiquitin-proteasome pathway proteins, interferon-related proteins or NF-κB-mediated cytokine/chemokine response proteins, such as MG-132 or ALLN may be administered orally, parenterally, sublingually, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrastemal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e. g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

In accordance with yet other embodiments, we provide methods for inhibiting any activity of ubiquitin-proteasome pathway proteins, interferon-related proteins or NF-κB-mediated cytokine/chemokine response proteins, in a human or animal subject, the method comprising administering to a subject an amount of a ubiquitin-proteasome pathway protein inhibitor compound which in some embodiments is MG-132 or ALLN (or composition comprising such compound) effective to inhibit the relevant activity in the subject. Other embodiments provide methods for treating dengue in a human or animal subject, comprising administering to the cell or to the human or animal subject an amount of a compound or composition as described here effective to inhibit a ubiquitin-proteasome pathway protein, interferon-related protein or NF-κB-mediated cytokine/chemokine response protein activity in the cell or subject. The subject may be a human or non-human animal subject. Inhibition of protein activity includes detectable suppression of the relevant protein activity either as compared to a control or as compared to expected protein activity.

Effective amounts of the inhibitors, agonists or antagonists of ubiquitin-proteasome pathway proteins, interferon-related proteins or NF-κB-mediated cytokine/chemokine response proteins, such as MG-132 or ALLN generally include any amount sufficient to detectably inhibit the relevant protein activity by any of the assays described herein, by other assays known to those having ordinary skill in the art or by detecting an alleviation of symptoms in a subject afflicted with dengue.

Successful treatment of a subject in accordance may result in the inducement of a reduction or alleviation of symptoms in a subject afflicted with a medical or biological disorder to, for example, halt the further progression of the disorder, or the prevention of the disorder. Thus, for example, treatment of dengue can result in a reduction in dengue associated symptoms such as fever, severe headache, joint and muscular pains (myalgias and arthralgias), rashes, gastritis, abdominal pain, nausea, vomiting, diarrhoea, haemorrhagic phenomena, thrombocytopenia, haemoconcentration or dengue shock syndrome (DSS).

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

A therapeutically effective dose will generally be from about 10 μg/kg/day to 100 mg/kg/day, for example from about 25 μg/kg/day to about 20 mg/kg/day or from about 50 μg/kg/day to about 2 mg/kg/day of an inhibitor, agonist or antagonist of a ubiquitin-proteasome pathway protein, an interferon-related protein or NF-κB-mediated cytokine/chemokine response protein, such as MG-132 or ALLN, which may be administered in one or multiple doses.

The inhibitors, agonists or antagonists of ubiquitin-proteasome pathway proteins, interferon-related proteins or NF-κB-mediated cytokine/chemokine response proteins, such as MG-132 or ALLN can also be administered in the form of liposomes. As is known in the art, liposomes are generally derived from phospholipids or other lipid substances. Liposomes are formed by mono-or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound, stabilizers, preservatives, excipients, and the like. Lipids which may be used include the phospholipids and phosphatidyl cholines (lecithins), both natural and synthetic. Methods to form liposomes are known in the art. See, for example, Prescott, Ed., Methods in Cell Biology, Volume XIV, Academic Press, New York, N. W., p. 33 et seq (1976).

While the inhibitors, agonists or antagonists of ubiquitin-proteasome pathway proteins, interferon-related proteins or NF-κB-mediated cytokine/chemokine response proteins, such as MG-132 or ALLN can be administered as the sole active pharmaceutical agent, they can also be used in combination with one or more other agents used in the treatment of disorders. Representative agents useful in combination with the inhibitors, agonists or antagonists of ubiquitin-proteasome pathway proteins, interferon-related proteins or NF-κB-mediated cytokine/chemokine response proteins, such as MG-132 or ALLN for the treatment of dengue include, for example, either of the P4-PMO compounds, 5′SL and 3′CS (targeting the 5′-terminal nucleotides and the 3′ cyclization sequence region, respectively) described in Kinney et al., 2005, Inhibition of dengue virus serotypes 1 to 4 in vero cell cultures with morpholino oligomers, J Virol. 79(8), 5116-28.

When additional active agents are used in combination with the inhibitors, agonists or antagonists of ubiquitin-proteasome pathway proteins, interferon-related proteins or NF-κB-mediated cytokine/chemokine response proteins, such as MG-132 or ALLN, the additional active agents may generally be employed in therapeutic amounts as indicated in the PHYSICIANS' DESK REFERENCE (PDR) 53rd Edition (1999), which is incorporated herein by reference, or such therapeutically useful amounts as would be known to one of ordinary skill in the art.

The inhibitors, agonists or antagonists of ubiquitin-proteasome pathway proteins, interferon-related proteins or NF-κB-mediated cytokine/chemokine response proteins, such as MG-132 or ALLN and the other therapeutically active agents can be administered at the recommended maximum clinical dosage or at lower doses. Dosage levels of the active inhibitors, agonists or antagonists of ubiquitin-proteasome pathway proteins, interferon-related proteins or NF-κB-mediated cytokine/chemokine response proteins, such as MG-132 or ALLN in the compositions may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions that are given at the same time or different times, or the therapeutic agents can be given as a single composition.

Bioavailability

The compounds disclosed here (and combinations) are in some embodiments orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. The inhibitors, agonists or antagonists of ubiquitin-proteasome pathway proteins, interferon-related proteins or NF-κB-mediated cytokine/chemokine response proteins, such as MG-132 or ALLN may in some embodiments have a minimum solubility of 50 mg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. The inhibitors, agonists or antagonists of ubiquitin-proteasome pathway proteins, interferon-related proteins or NF-κB-mediated cytokine/chemokine response proteins, such as MG-132 or ALLN may have a Log D_{7.4} of −2 to +4 or −1 to +2. The log D can be determined by standard procedures known in the art such as described in J. Pharm. Pharmacol. 1990, 42:144.

Cell monolayer assays such as CaCO₂ add substantially to prediction of favourable membrane permeability in the presence of efflux transporters such as p-glycoprotein, so-called caco-2 flux. The inhibitors, agonists or antagonists of ubiquitin-proteasome pathway proteins, interferon-related proteins or NF-κB-mediated cytokine/chemokine response proteins, such as MG-132 or ALLN may have a caco-2 flux of greater than 2×10⁻⁶ cms⁻¹, for example greater than 5×10⁻⁶ cms⁻¹. The caco flux value can be determined by standard procedures known in the art such as described in J. Pharm. Sci, 1990, 79, 595-600.

Metabolic stability addresses the ability, of the GIT or the liver to metabolise compounds during the absorption process: the first pass effect. Assay systems such as microsomes, hepatocytes etc are predictive of metabolic liability. The compounds of the Examples may in some embodiments show metabolic stability in the assay system that is commensurate with an hepatic extraction of less than 0.5. Examples of assay systems and data manipulation are described in Curr. Opin. Drug Disc. Devel., 201, 4, 36-44, Drug Met. Disp.,2000, 28, 1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp.,1999, 27, 221-226.

The term “pharmaceutically acceptable carrier” as used herein generally refers to organic or inorganic materials, which cannot react with active ingredients. The carriers include but are not limited to sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oil, cotton seed oil, sesame oil, olive oil, corn oil and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations. Wetting agents and lubricants such as sodium lauryl sulfate, as well as coloring agents, flavoring agents, lubricants, excipients, tabletting agents, stabilizers, anti-oxidants and preservatives, can also be present.

The term “therapeutically effective amount” as used herein generally. refers to an amount of an agent, for example the amount of a compound as an active ingredient, that is sufficient to effect treatment as defined herein when administered to a subject in need of such treatment. A therapeutically effective amount of a compound, salt, derivative, isomer or enantiomer of the present invention will depend upon a number of factors including, for example, the age and weight of the subject, the precise condition requiring treatment and its severity, the nature of the formulation, and the route of administration, and will ultimately be at the discretion of the attendant physician or veterinarian. However, an effective amount of a compound of the present invention for the treatment of disorders associated with bacterial or viral infection, in particular bacterial meningitis, will generally be in the range of about 10 to about 40 mg/kg body weight of recipient (mammal) per day and more usually about 40 mg/kg body weight per day. Thus, for a 70 kg adult subject, the actual amount per day would typically be about 2,800 mg, and this amount may be given in a single dose per day or more usually in a number (such as two, three, four, five or six) of sub-doses per day such that the total daily dose is the same. An effective amount of a salt of the present invention may be determined as a proportion of the effective amount of the compound per se.

The term “treatment” as used herein refers to any treatment of a condition or disease in an animal, particularly a mammal, more particularly a human, and includes: preventing the disease or condition from occurring in a subject which may be predisposed to the disease but has not yet been diagnosed as having it; inhibiting the disease or condition, i.e. arresting its development; relieving the disease or condition, i.e. causing regression of the condition; or relieving the conditions caused by the disease, i.e. symptoms of the disease.

Chemical Derivative

The term “derivative” or “derivatised” as used herein includes chemical modification of a compound. Illustrative of such chemical modifications would be replacement of hydrogen by a halo group, an alkyl group, an acyl group or an amino group.

Chemical Modification

In one embodiment, the compound may be a chemically modified compound.

The chemical modification of a compound may either enhance or reduce hydrogen bonding interaction, charge interaction, hydrophobic interaction, Van Der Waals interaction or dipole interaction between the compound and the target.

In one aspect, the identified compound may act as a model (for example, a template) for the development of other compounds.

Individual

The compounds are delivered to individuals. As used herein, the term “individual” refers to vertebrates, particularly members of the mammalian species. The term includes but is not limited to domestic animals, sports animals, primates and humans.

### Other Uses

Use of the Identified Targets in the Ubiquitin or Interferon Pathways (Above) for Developing Therapeutic Treatments for Dengue.

Development of therapeutic treatment for dengue can be done by a process of screening substances for their ability to interact with a ubiquitin or interferon pathway target (listed above) polypeptide or a gene transcription regulatory polypeptide, the process comprising the steps of providing a polypeptide described here and testing the ability of selected substances to interact with that polypeptide.

Utilizing the methods and compositions described here, screening assays for the testing of candidate substances such as agonists and antagonists can be derived. A candidate substance is a substance which can interact with or modulate, by binding or other intramolecular interaction, a ubiquitin or interferon pathway member polypeptide or a gene transcription regulatory polypeptide. In some instances, such a candidate substance is an agonist and in other instances can exhibit antagonistic attributes when interacting with the receptor polypeptide. In other instances, such substances have mixed agonistic and antagonistic properties or can modulate the pathway in other ways. Alternatively, such substances can promote or inhibit transcription of the pathways.

Screening assays may generally involve determining the ability of a candidate substance to bind to a pathway member and to affect the activity of the pathway, such as the screening of candidate substances to identify those that inhibit or otherwise modify the pathway's function. Typically, this method includes preparing potential therapeutic recombinant substance, followed by testing to determine the ability of the substance to affect the pathway's function. In some embodiments, we describe the screening of candidate substances to identify those that affect the activity of the pathway, in a similar way to the demonstrations in this application using the ubiquitin pathway inhibitors MG-132 and ALLN.

As is well known in the art, a screening assay provides the conditions suitable for the binding of an agent to members of the ubiquitin or interferon pathways. These conditions include but are not limited to pH, temperature, tonicity, the presence of relevant co-factors, and relevant modifications to the polypeptide such as glycosylation, palmytoilation, or prenylation. pH may be from about a value of 6.0 to a value of about 8.0, such as from about a value of about 6.8 to a value of about 7.8 and, or about 7.4. In a embodiment, temperature is from about 20.degrees C. to about 50.degrees C., such as from about 30.degrees C. to about 40.degrees C. or about 37.degrees C. Osmolality may be from about 5 milliosmols per liter (mosm/L) to about 400 mosm/1 and, such as from about 200 milliosmols per liter to about 400 mosm/l or from about 290 mosm/L to about 310 mosm/L. Typical co-factors include sodium, potassium, calcium, magnesium, and chloride. In addition, small, non-peptide molecules, known as prosthetic groups can be required. Other biological conditions needed for function are well known in the art.

Accordingly, it is proposed that this disclosure provides those of skill in the art with methodology that allows for the identification of candidate substances having the ability to modify the action of the ubiquitin or interferon pathways in one or more manners and can exert their physiological effects through a secondary molecule.

In that most such screening assays are designed to identify agents useful in mimicking the desirable aspects of the examples given (demonstrations in this application using the ubiquitin pathway inhibitors) while eliminating the undesirable aspects.

There are believed to be a wide variety of embodiments that can be employed to determine the effect of the candidate substance on a pathway described gene, and the invention is not intended to be limited to any one such method. However, it is generally desirable to employ a system wherein one can measure the ability of the candidate to affect the pathway, and the outcome on viral replication, as shown in detail above.

The detection of an interaction between an agent, pathway and viral replication can be accomplished through techniques well known in the art.

Use of the Identified Targets in the Ubiquitin or Interferon or NF-κB-Mediated Cytokine/Chemokine Response Protein as a Method of Providing an Indication Useful in the Diagnosis of Dengue.

A point-of-care immunoassay dipstick test to quantify host factors for the dengue viremia stage as well as the convalescence stage will greatly benefit clinicians in managing patients, and also serve as markers to indicate the disease course when applying treatments as they become available.

The test could involve a “dipstick”, a solid matrix where the serum sample can be applied at the bottom chamber A (serum sample application chamber). The dip stick could have chamber B (test chamber) spotted with reagents to capture dengue virus specific antigen such as NS1 and at least two other host factors such as IP-10 and/or I-TAC as well as other markers identified in our study. The mobile phase for migration of the antigens to reach chamber B and C can be added to chamber A. The presence of the factors under test can be detected by colored reporter agents that will make the spots in chamber B and C (positive control chamber) visible if the antigens are present in the serum. If the antigens are absent then only the spots in chamber C should be visible. The colored detector agents may be antibodies that carry a visible reporter. If the antigen is absent in the serum then antigen that is embedded in the space between chamber B and C can be moved by capillary action to chamber C where the capture reagent will bind the antigen together with the colored reporter agent to indicate that the test is in working order.

### Examples

### Example 1

### Host Factors Involved in Dengue Replication: Differential Expression of a Gene Cluster in the Ubiquitin-Proteasome Pathway in Infected Cells

In order to identify these host factors involved in dengue replication, we have undertaken an analysis of the changes in gene expression in a human hepatocytic cell line following infection with dengue virus (HepG2, ATCC#HB-8065).

The HepG2 cell line is infected with TSV01, a serotype 2 dengue virus (Genbank AY037116; McBride and Vasudevan, 1995), and cells harvested over a succession of time points up to three days post-infection.

We monitored viral replication using PCR and Plaque assay techniques. Comparisons between live virus infection and heat-inactivated virus are used to provide information about the host factors involved in virus replication.

Furthermore, we analyzed the changes in host response using a GIS in-house microarray presenting 19,800 Compugen oligos and compared the relative expression of the host compared to a universal reference sample (Stratagene).

Genes that are significantly differentially expressed between live and heat inactivated virus are determined using a “statistical analysis of microarray” (SAM) approach.

Detailed manual analysis of this list identified a gene cluster, in the ubiquitin-proteasome pathway, that is differentially expressed (upregulated) in cells with viral replication. The ubiquitin-proteasome pathway is shown in FIG. 1

### Example 2

### Inhibition of Viral Replication By Compounds Which Affect the Function of the ubiquitin-proteasome Pathway (Time Course)

In a second experiment we applied specific compounds to our in vitro experiment (described above) that are known to alter the function of these (and other) genes in this pathway (Table E1 below).

TABLE E1
Compounds known to have inhibitory effects on the ubiquitin-proteasome
pathway.

| Compound, provider | Formula | Proposed effect |
|---|---|---|
| Ammonium | C₅H₉NS₂•NH₃ | Inhibit E3 ubiquitin ligase or directly |
| pyrrolidinedithiocarbamate | | inhibit proteasome activity. |
| (PDTC) Sigma | | |
| Carbobenzoxy-L-leucyl-L- | C₂₆H₄₁N₃O₅ | Reduces the degradation of ubiquitin- |
| leucyl-L-leucinal (MG-132) | | conjugated proteins in mammalian cells |
| Merck | | by the 26S complex without |
| (FIG. 2) | | affecting its ATPase or isopeptidase |
| | | activities. |
| N-Acetyl-Leu-Leu-Nle-CHO | C₂₀H₃₇N₃O₄ | Inhibits the proteolysis I\| B-α and |
| (ALLN) Merck | | I\| B-β by the ubiquitin-proteasome |
| | | complex. |

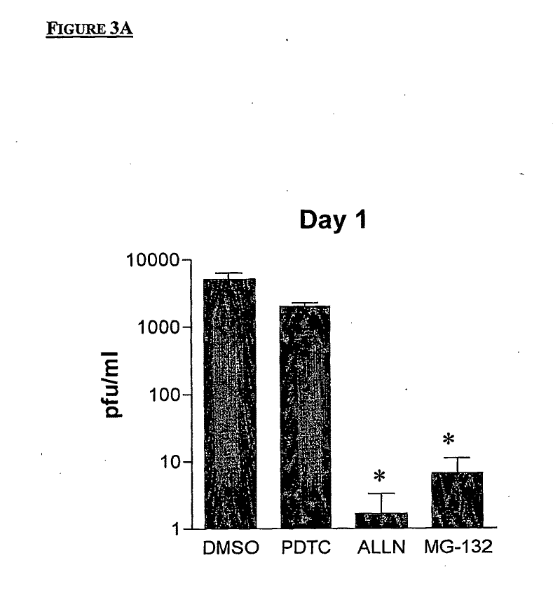

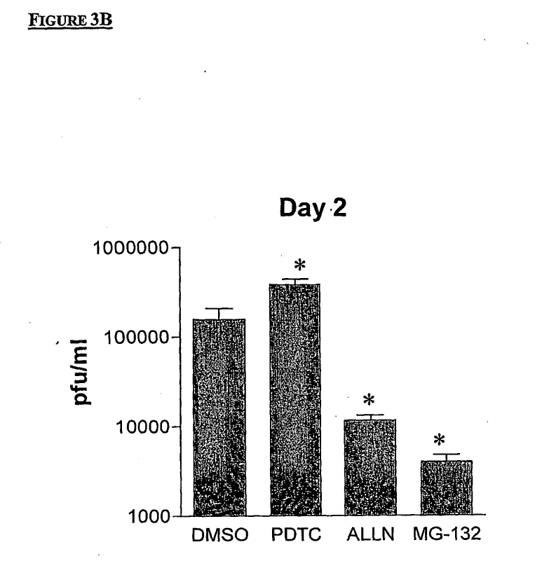

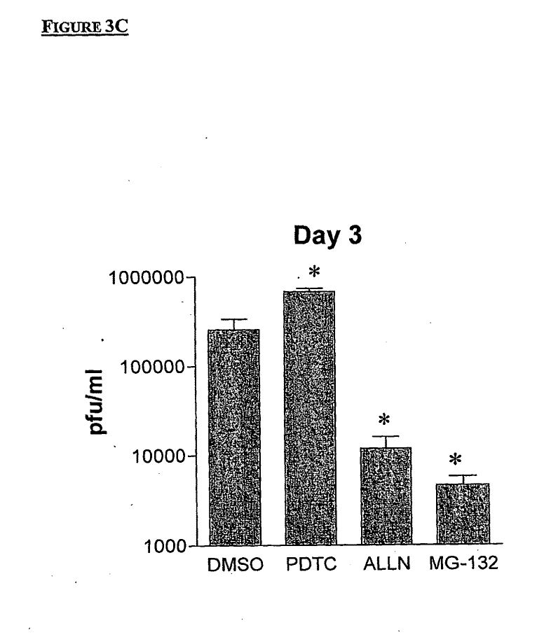

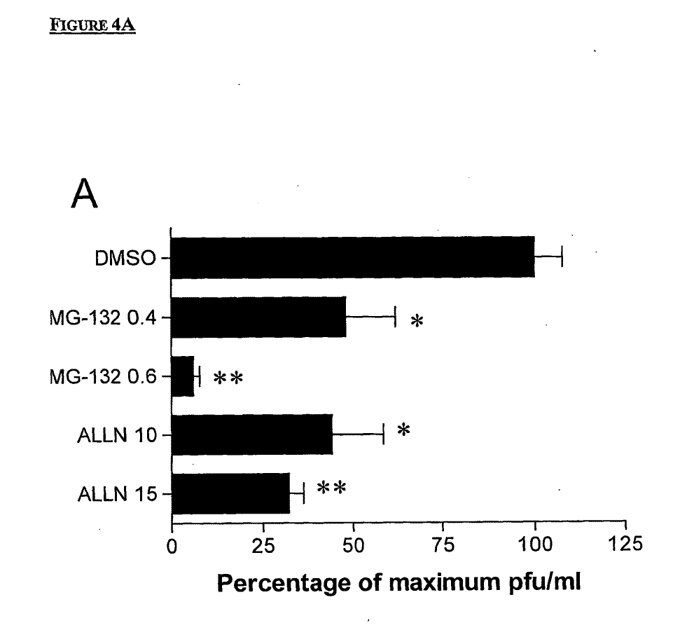

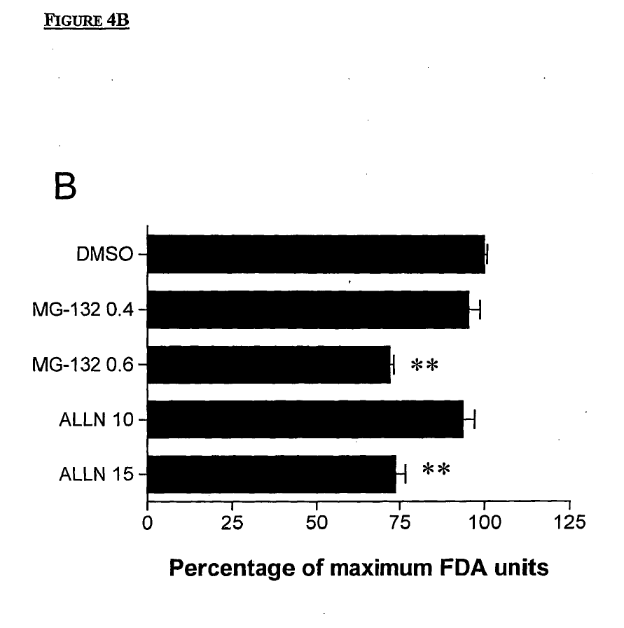

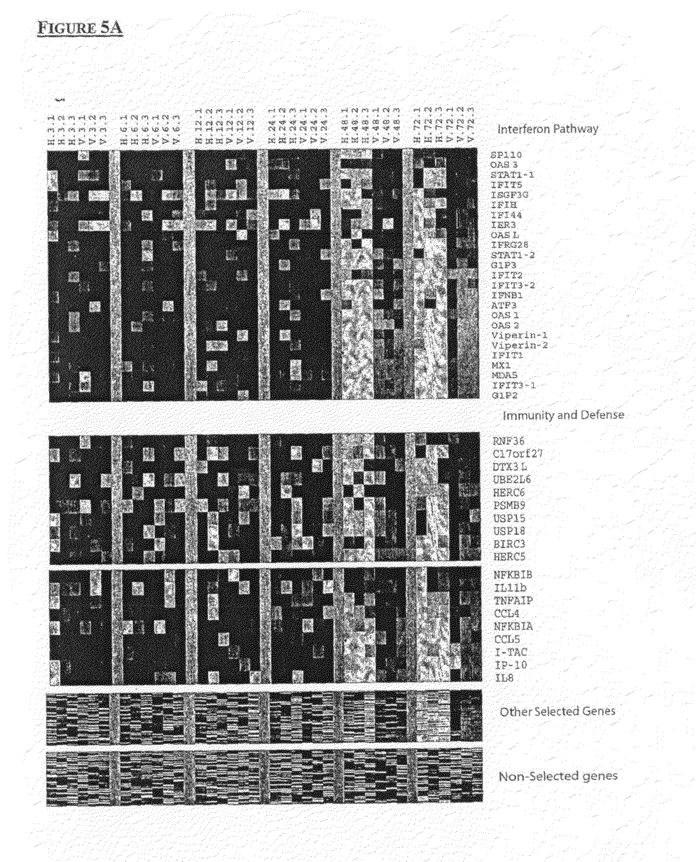

Analysis of viral replication following the addition of these compounds showed that a significant reduction in viral replication occurred, indicating that these genes are important controllers of viral replication (FIGS. 3A, 3B and 3C).

Viral load in the early phase of dengue infection has been shown to correlate to disease severity, and a therapeutic intervention reducing virus replication has potential to prevent DHF and subsequent mortality (Vaughn et al., 2000).

### Example 3

### Concentration Dependent Effect of Compounds That Inhibit the Ubiquitin Pathway on Dengue Viral Production

HepG2 cells (ATCC) are treated with proteasome inhibitors prior to infection with dengue virus. The following inhibitors are used:
MG-132 (Carbobenzoxy-L-leucyl-L-leucyl-L-leucinal, Merck) at 0.4 μM and 0.6 μM in DMSO—see FIG. 2
ALLN (N-Acetyl-Leu-Leu-Nle-CHO, Merck) at 10 μM and 15 μM in DMSO

DMSO is used as reference is these experiments. A FDA assay is used to assess specific cytotoxicity exerted by the compounds.

The protocol is as follows:

HepG2 cells are cultured ON in 24 well plates. Cells are incubated with MG-132 (0.4 μM and 0.6 μM in DMSO) and ALLN (10 μM and 15 μM in DMSO) and with DMSO alone for 2 hrs prior to infection with dengue virus TSV01 (MOI 10) for 48 hrs.

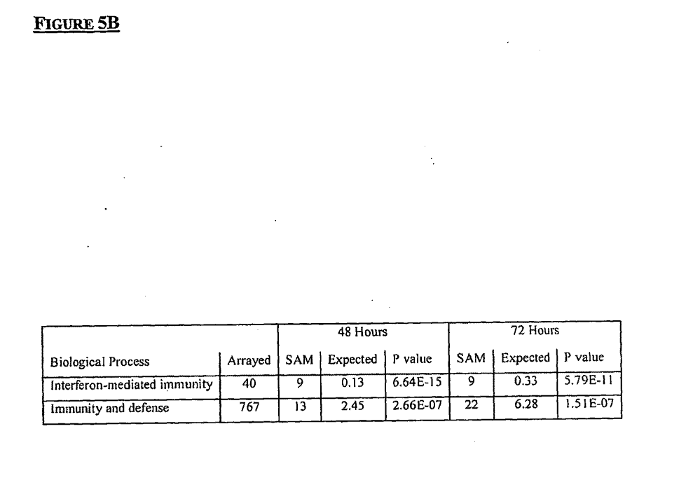

Cell culture supernatants are collected and assayed for dengue virus by plaque assay. Plaque forming units per ml are expressed as the mean percentage of the highest number of pfu/ml (DMSO alone)±SEM, (n=3). Cytotoxicity is measured using FDA and results are expressed as the mean percentage compared to DMSO alone±SEM, (n=3). “*” indicates p<0.05 and “**”indicates p<0.005 by students t-test comparing each treatment to DMSO alone. The results are shown in FIG. 4A and FIG. 4B.

MG-132 and ALLN treatment significantly reduced virus replication in the HepG2 cell line as shown by plaque assay of the cell culture supernatants two days after infection (FIG. 4A).

An examination of the effects of these compounds on the cells using FDA revealed a degree of cytotoxicity, 5% to 28% over the concentrations of compound tested, compared to the 52% to 94% reduction in plaque forming units (FIG. 4B).

### Example 4

### Expression of Ubiquitin-Proteasome Pathway Genes in Early Phase of Dengue Infected Patients

In a further experiment, we noted that the genes identified above are highly active in the blood of patients with dengue during the critical early phase of dengue, when viral replication is at its peak. This finding confirms that the readout from the in vitro assay translates to the in vivo host-response.

We predict that a compound able to effectively inhibit the described genes will reduce viral replication in patients and reduce disease severity, offering a treatment for Dengue.

### Example 5

### Confirmation of Ubiquitin Pathway Activation in a Second Cell Line and Patients with Dengue

From an initial experiment using a microarray approach to discover novel genes affecting dengue viral replication, a candidate list of 11 ubiquitin pathway genes are identified that are altered in the HepG2 cell line during dengue virus replication.

We measured the activation of these genes using a Taqman PCR approach in the HepG2 cell line (as described above) and in a similar manor in a second cell line (A549). We also measured the expression of these genes in 15 dengue patients at an early stage of active disease compared to 3 to 4 weeks later when they are in recovery.

We identified 3 of these 11 genes to be significantly up regulated by dengue viral replication in all three conditions—see Table E2 below.

TABLE E2
Ubiquitin pathway genes found responding to dengue
viral replication in patients and cell lines.

| | | Gene | GenBank | Chromosomal |
|---|---|---|---|---|
| No | Name | Symbol | Accession No | Location |
| 1 | ISG15 IFI15 | G1P2 | NM_005101 | 1p3633/G1P2 |
| 2 | E3 Ub ligase | HERC5 | NM_016323 | 4q221q23/HERC5 |
| 3 | ISG15 cleavage | USP18 | NM_017414.2 | 22q1121/USP18 |

### Example 6

### Methods

Cell Culture

All cell lines are obtained from ATCC and maintained in RPMI 1640 (BHK-21, THP-1, C6/36), Minimal Essential Medium (Vero, HepG2, HeLa, SK Hep-1, JAWSII), Hams F12K (, HUV-EC-C) or Dulbecco's Modified Eagle Medium (293T/17, J774A.1, RAW264.7, A549, A549-viperin) cell culture medium (GibcoBRL) with 10% fetal bovine serum (FBS) and penicillin/streptomycin (GibcoBRL). All cells are cultured at 37° C. in a humidified incubator with 5% (v/v) CO₂, except for C6/36 cells which are cultured at 28° C. Cytotoxicity is monitored using FDA.

Virus Culture, Infection and Assaying

The type 2 dengue virus strain TSV01 is obtained from an outbreak in Townsville, Australia (McBride and Vasudevan 1995) (GenBank, accession number AY037116). TSV01 is cultured in the C6/36 cell line (from the Aedes albopictus mosquito) with virus added to cells in a T175 flask (MOI 0.01) and incubated for 1 hr at 28° C. with brief mixing every 20 min. Media is removed and cells washed in fresh media then media with 5% FBS added and cells incubated for 5 days at 28° C. Cell culture supernatants are removed and centrifuged at 1000×g for 10 min at 4° C. then pooled and stored in aliquots at −80° C. Heat-inactivated virus is prepared by incubating virus samples at 55° C. for 1 hr, with heat-inactivation confirmed by plaque assay.

For plaque assay, BHK-21 cells are cultured ON in 24 well plates before media is removed and serial dilutions (10-fold) of cell culture supernatants added to individual wells. Plates are incubated for 1 hr before media is aspirated and replaced with 0.5 ml of 0.8% methyl-cellulose medium (with 2% FBS). Plates are then incubated for 5 days before the media is removed and cells fixed in 4% formaldehyde for 20 min then rinsed in water and stained with crystal violent for 20 min and rinsed again. Plaques are counted manually and concentrations of plaque forming units per ml (pfu/ml) of the sample cell culture supernatant calculated.

Selection of an In Vitro Infection System

Thirteen mammalian cell lines are screened for their ability to support infection and replication of dengue virus. These included hamster (BHK-21) and monkey (Vero) cells, mouse monocytic cell lines (JAWSII, RAW264.7, J774A.1) and human epithelial (HepG2, A549, Hela, 293T/17), endothelial (SK Hep-1, HUV-EC-C) and monocytic (K562, THP-1) cell lines. Cells are cultured in 24 well plates and infected with dengue virus TSV01, at an MOI of 1 or 10 for 24, 48 and 72 hrs. Cell culture supernatants are removed and stored at −80° C. before virus concentrations are determined by plaque assay.

Generation of A549 Cells Stably Expressing Viperin and Treatment with IFN-β

A549 cells are transfected with an expression construct encoding viperin by lipofectamine 2000 (InVitrogen). Cells are selected using 500 μg/m1 G418 and screened for viperin expression by immunoblotting with anti-viperin antibody. Cells are cultured ON in 6 well plates before IFN-β (IMCB, Singapore), at a final concentration of 500 U/ml, is added to each well while control wells remained untreated. Twelve hours post IFN-β-treatment, cell culture medium is removed, replaced with fresh medium, and the cells infected with dengue virus (TSV01; MOI 1) for 48 hrs before cell culture supernatants are removed and the number of plaque forming units determined by plaque assay.

Chemokine Assays

Protein concentrations in cell culture supernatants and patient serum samples are assayed by commercial ELISA as per manufacturers instructions for IP-10 and I-TAC (R&D Systems).

Flow Cytometry

Dengue E-protein is assessed in suspension HepG2 cells by intracellular staining and fluorescence activated cell sorter (Becton Dickinson) and an Alexa-647 conjugated (AlexaFluor conjugation kit) monoclonal antibody (4G2, ATCC). Cells are permeabilized using BD FACS Perm/Wash solution (BD) and acquisition and analysis is performed using CellQuest software (BD).

Patient Samples

Adults with clinical symptoms consistent with dengue and fever duration of less than 72 hrs are sampled for serum (plain tubes) and whole blood (PAXgene vacutainer tube, Qiagen, X) at a Singapore primary healthcare clinic. Serum samples are analyzed by real-time PCR for presence of dengue virus 1-4 RNA (Taiwan, CDC). PCR positive individuals are prospectively included in the study and underwent repeat sampling 3 to 4 weeks after initial fever presentation.

RNA Processing

RNA is extracted from cultured cells using the RNeasy Mini Kit (QIAGEN, Germany). For patient blood samples collected in PAXgene tubes RNA is extracted using PAXgene Blood RNA Kit (PreAnalytiX). RNA is subjected to DNase treatment using RNase-Free DNase Set (QIAGEN, Germany).

Microarray

Human 19k oligonucleotide arrays (representing 18861genes) are manufactured by spotting 60 mer oligonucleotide probes (designed by compugen and manufactured by Sigma-Genosys) onto poly-L-lysine-coated microscope slides using GeneMachines OmniGrid Microarray Spotter in the Genome Institute of Singapore. The printed arrays are post-processed according to the standard protocol described for cDNA microarray (Eisen et al 1999).

For fluorescence labeling of target cDNAs, 20 μg of total RNA from universal human reference (Strategene. USA) and experiment samples are reverse transcribed in the presence of Cy3-dUTP and Cy5-dUTP (Amersham Biosciences, Little Chalfont, UK) by using the Superscript reverse transcription kit (Invitrogen, USA). Labeled cDNA are pooled, concentrated, re-suspended in DIG EasyHyb (Roche, Basel, Switzerland) buffer and hybridized overnight (14-16 h) in the MAUI Hybridization chamber (BioMicro, Salt Lake City, Utah). The arrays are scanned using a GenePix 4000B Scanner (Axon Instruments, USA) to generate Tiff images. The images are analyzed by GenePix Pro 4.0 software (Axon Instruments, USA) to measure Cy3 and Cy5 fluorescence signals intensity and format data for data base deposition.

For every sample at all time points dye swap is performed and a rigorous quality check is done before an array is used for down stream analysis (Miller et al 2002). The array data then undergoes lowess normalization available in an R package aroma to remove channel specific biases (Bengtsson et al 2004, R Development Core Team 2005). Further quality of the data is assured by computing the correlation of technical repeats. For all but one of the technical repeats we obtained a correlation above 0.85.

Selection of Differentially Expressed Genes from Microarray Data

Differentially expressed genes are selected using a procedure known as Significance Analysis of Microarrays ((SAM), Tusher et al 2001), described in brief below: The statistic used in SAM is given as

$$d = \frac{{μ}_{1} - {μ}_{2}}{s + {s}_{0}}$$

where; the numerator is the group mean difference, s the standard error, and so a regularizing constant. Setting so=0 will yield a t-statistic. This value, called the fudge constant, is found by removing the trend in d as a function of s in moving windows across the data to reduce false positive results [Chu et al 2005]. As the statistic is not t-distributed significance is computed using a permutation test. Genes with a computed statistic larger than the threshold are called significant. The false discovery rate (FDR) associated with the given threshold can also be calculated from the permutation data.

TaqMan Low Density Array (TLDA)

100 ng of total RNA is reverse transcribed using High-Capacity cDNA Archive Kit (ABI). Reverse-transcriptase reaction is performed at 25° C. for 10 min and then 37° C. for 2 hours. 1 μg of cDNA in 50 μl buffer is added to 50 μl TaqMan Universal Master Mix (2×) (ABI), immediately loaded into a Micro Fluidic Card (3M Company, ABI) that is spun twice at 1200 rpm for 1 min each time to distribute the PCR mix into the wells of the card, before sealing and loading into the ABI 7900HT. Default thermal cycling conditions are used (50° C. for 2 min with 100% ramping, 94.5° C. for 10 min with 100% ramping and finally, 40 cycles of 97° C. for 30 sec with 50% ramping and 59.7° C. for 1 min with 100% ramping) and data analyzed using SDS2.2 software (ABI). As there are three biological replicates in cell culture experiments and 10 replicates in the patient studies we are able to use the same SAM procedure described above to select differentially expressed genes in the TLDA results.

Pathway Detection

We analyzed SAM gene lists for pathway information using the Applied Biosystem online program “panther” gene expression analysis systems (http://www.pantherdb.org/). “Panther” can subdivide large collection of proteins or genes into functional (ontology terms and pathway) relationships in a robust and accurate way (Huaiyu et al 2005). We also used an additional method for pathway detection.

### Example 7

### Cell and Strain Selection

We screened thirteen mammalian cell lines for their ability to support replication of different dengue virus strains. We identified the clinical, dengue serotype 2 isolate TSV01 as the most readily replicating in our cell lines and infected all cell lines for 24, 48 and 72 hrs.

Cell lines are ranked by maximum pfu/ml titer produced; Vero>BHK-21>A549>HepG2>SK-Hep1>K562>JAWSII>293T/17>HUV-EC-C>THP-1>J774A.1>RAW264.7>HeLa. The highest yielding human cell lines (A549 and HepG2) are used in further studies, with HepG2 as the initial focus because of evidence of dengue in the liver (the source of the HepG2 cell line) during infection (Jessie, Fong et al. 2004).

### Example 8

### Expression Analysis: FACS and PCR/Microarray

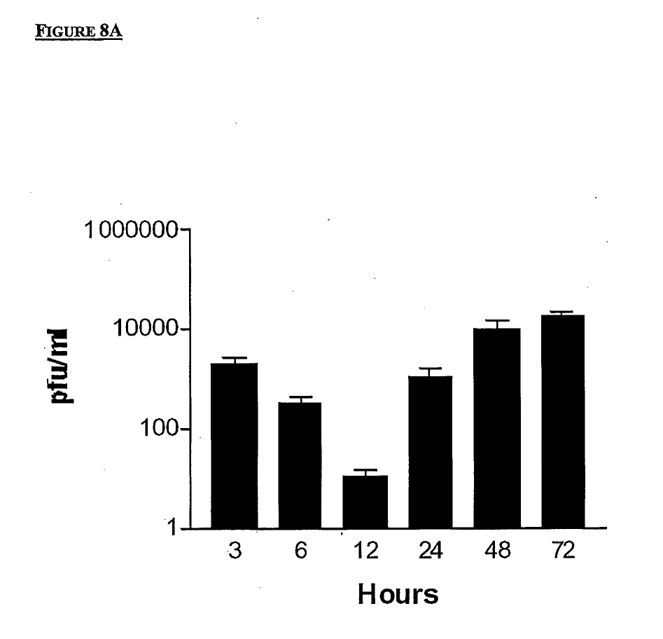

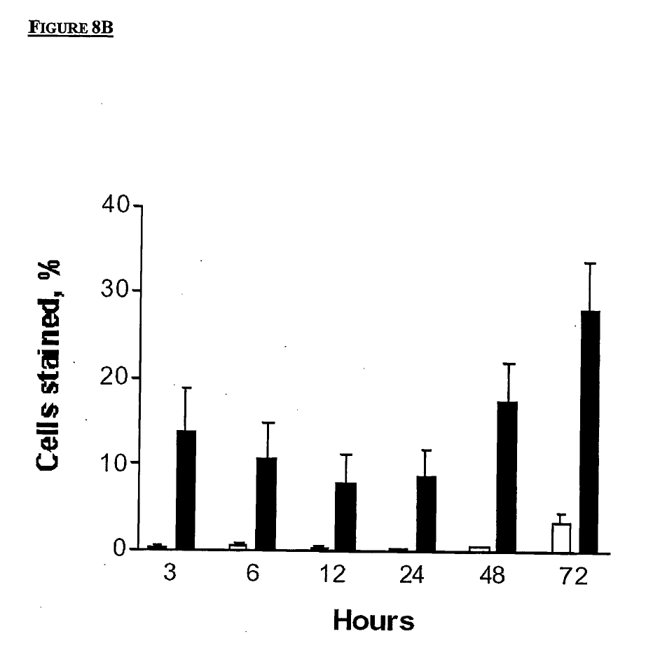

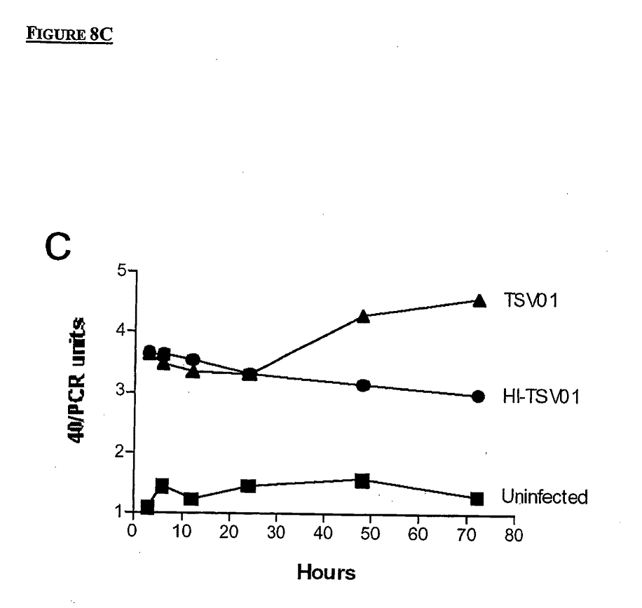

Viral replication in HepG2 cells infected with dengue virus TSV01 for 3, 6, 12, 24, 48 and 72 hrs, compared to heat inactivated virus, is determined by plaque assay, FACS analysis and real-time PCR analysis (FIG. 8A, FIG. 8B and FIG. 8C).

All three methods showed that new viral replication began after 24 hours and peaked at 72 hours. Analysis of microarrays (performed in duplicate on three biological replicates at each time point, comparing infectious and heat inactivated virus, 72 slides) using SAM (Statistical Analysis of Microarray), revealed no significantly differentially expressed genes at 3, 6, 12 and 24 hrs.

There are 24 transcripts identified at 48 hrs and 124 at 72 hrs. The combined list of 132 transcripts (124 genes) clustered neatly into interferon pathway and immunity and defense by Panther pathway analysis (Huaiyu et al 2005) (FIG. 5A, FIG. 5B and Table E3 below).

TABLE E3
132 transcripts, 124 genes, 4 groups

| | Accession | | | 48 Hours | | 72 Hours | |
|---|---|---|---|---|---|---|---|
| Gene Name | number | Group | Gene descriptionff | qV | F.C. | qV | F.C. |
| CCL4 | NM_002984 | Chemokine | Cytokine and chemokine mediated | 10.9 | 1.6 | 3.0 | 2.2 |
| | | | signaling pathway; | | | | |
| IL11b | NM_000881 | Chemokine | interleukin 11 (IL11b) mRNA | — | — | 7.0 | 1.7 |
| IL8 | M17017 | Chemokine | Chemokine activity, attracts | 3.8 | 4.4 | 3.0 | 6.6 |
| | | | neutrophils, basophils, and t-cells | | | | |
| IP-10 | NM_001565 | Chemokine | Cytokine and chemokine | 3.8 | 2.0 | 7.0 | 3.4 |
| | | | signaling; Macrophage-mediated | | | | |
| | | | immunity | | | | |
| I-TAC | NM_005409 | Chemokine | Cytokine and chemokine | 5.4 | 1.8 | 7.0 | 2.8 |
| | | | signaling; Macrophage-mediated | | | | |
| | | | immunity | | | | |
| NFKBIA | NM_020529 | Chemokine | mRNA transcription; NF-kappaB | 10.9 | 1.7 | 3.0 | 2.5 |
| | | | cascade; Intracellular protein traffic | | | | |
| NFKBIB | NM_002503 | Chemokine | mRNA transcription; NF-kappaB | — | — | 23.1 | 1.4 |
| | | | cascade; Intracellular protein traffic | | | | |
| RANTES | NM_002985 | Chemokine | Cytokine and chemokine mediated | 3.8 | 2.5 | 3.0 | 2.5 |
| | | | signaling pathway; | | | | |
| TNFAIP | NM_006290 | Chemokine | tumor necrosis factor, alpha-induced | — | — | 12.3 | 1.9 |
| | | | protein 3 (TNFAIP3) mRNA | | | | |
| ATF3 | NM_004024 | Interferon | mRNA transcription | 5.4 | 2.1 | 4.1 | 3.3 |
| | | | regulation; Induction of apoptosis | | | | |
| G1P3 | NM_002038 | Interferon | interferon, alpha-inducible protein | 3.8 | 2.6 | 3.0 | 2.3 |
| | | | (clone IFI-6-16) | | | | |
| IER3 | NM_003897 | Interferon | immediate early response 3 | — | — | 12.3 | 2.0 |
| IFI44 | NM_006417 | Interferon | interferon-induced, hepatitis C- | — | — | 3.0 | 2.0 |
| | | | associated microtubular aggregate | | | | |
| | | | protein | | | | |
| IFIH1 | AL080107 | Interferon | RNA helicase, DEAD box protein, | 3.8 | 2.1 | 23.1 | 1.9 |
| | | | upregulated with beta-interferon | | | | |
| IFIT1 | NM_001548 | Interferon | interferon-induced protein with | 3.8 | 4.3 | 3.0 | 5.8 |
| | | | tetratricopeptide repeats 1; IFIT1 | | | | |
| IFIT2 | AF026944 | Interferon | Interferon-induced protein with | — | — | 7.0 | 2.6 |
| | | | tetratricopeptide repeats 2 | | | | |
| IFIT3(1) | AF026943 | Interferon | Interferon-induced protein with | 3.8 | 5.5 | 3.0 | 7.9 |
| | | | tetratricopeptide repeats 3 | | | | |
| IFIT3(2) | NM_001549 | Interferon | interferon-induced protein with | 3.8 | 2.6 | 3.0 | 2.6 |
| | | | tetratricopeptide repeats 3 | | | | |
| IFIT5 | NM_012420 | Interferon | retinoic acid- and interferon-inducible | 10.9 | 1.6 | — | — |
| | | | protein (58 kD) (RI58) | | | | |
| IFNB1 | NM_002176 | Interferon | interferon, beta 1, fibroblast | 3.8 | 3.2 | 3.0 | 3.1 |
| IRF9 | NM_006084 | Interferon | mRNA transcription | 5.4 | 2.0 | 12.3 | 1.7 |
| | | | regulation; Interferon-mediated | | | | |
| | | | immunity | | | | |
| IFRG28 | AJ251832 | Interferon | 28 kD interferon responsive protein | 3.8 | 1.9 | 4.1 | 2.2 |
| ISG15 | NM_005101 | Interferon | Proteolysis, interferon-stimulated | 3.8 | 11.3 | 3.0 | 9.2 |
| | | | protein, 15 kDa myxovirus (influenza | | | | |
| | | | virus) resistance 1, interferon-inducible | | | | |
| | | | protein p78 | | | | |
| MxA | NM_002462 | Interferon | nucleotide and nucleic acid | 3.8 | 7.3 | 3.0 | 5.0 |
| | | | metabolism; Interferon-mediated | | | | |
| | | | immunity | | | | |
| OAS1 | NM_016816 | Interferon | nucleotide and nucleic acid | 3.8 | 4.4 | 3.0 | 3.6 |
| | | | metabolism; Interferon-mediated | | | | |
| | | | immunity | | | | |
| OAS2 | NM_002535 | Interferon | Nucleic acid | 3.8 | 5.6 | 3.0 | 4.0 |
| | | | binding; Nucleotidyltransferase; Defense/ | | | | |
| | | | immunity protein | | | | |
| OAS3 | NM_006187 | Interferon | | — | — | 23.1 | 1.4 |
| OASL | AF063611 | Interferon | 2′-5′oligoadenylate synthetase-related | 10.9 | 1.7 | 3.0 | 2.1 |
| | | | protein p56 (OASL) | | | | |
| MDA5 | AF095844 | Interferon | interferon induced with helicase C | 3.8 | 4.7 | 3.0 | 7.0 |
| | | | domain 1; IFIH1 | | | | |
| SP110 | NM_004510 | Interferon | interferon-induced protein 75, 52 kD | 3.8 | 2.0 | — | — |
| | | | (IFI75) mRNA transcription | | | | |
| STAT1(1) | AK022231 | Interferon | signal transducer and activator of | 3.8 | 1.8 | — | — |
| | | | transcription | | | | |
| STAT1(2) | NM_007315 | Interferon | signal transducer and activator of | 3.8 | 2.9 | 3.0 | 2.2 |
| | | | transcription | | | | |
| VIPERIN(1) | AF026941 | Interferon | Virus inhibitory, endoplasmic reticulum- | 3.8 | 3.4 | — | — |
| | | | associated, interferon inducible | | | | |
| VIPERIN(2) | AF026942 | Interferon | Virus inhibitory, endoplasmic reticulum- | 3.8 | 3.8 | 3.0 | 3.9 |
| | | | associated, interferon inducible | | | | |
| BIRC3 | NM_001165 | Ubiquitin | Ubiquitin-protein ligase activity, Anti- | — | — | 4.1 | 2.0 |
| | | | apoptosis | | | | |
| C17orf27 | AB046774 | Ubiquitin | Ubiquitin-protein ligase activity | 5.4 | 1.8 | — | — |
| DTX3L | AK025135 | Ubiquitin | Ubiquitin-protein ligase activity | 5.4 | 1.8 | 23.1 | 1.5 |
| HERC5 | NM_016323 | Ubiquitin | Ubiquitin-protein ligase, cyclin-E | 3.8 | 6.0 | 3.0 | 10.6 |
| | | | binding protein 1 | | | | |
| HERC6 | NM_017912 | Ubiquitin | Ubiquitin-protein ligase | — | — | 23.1 | 1.6 |
| PSMB9 | NM_002800 | Ubiquitin | proteasome (prosome, macropain) | 7.7 | 1.8 | 4.1 | 1.7 |
| | | | subunit, beta type, 9 | | | | |
| RNF36 | AL360161 | Ubiquitin | Ubiquitin-protein ligase activity, | 5.4 | 1.7 | — | — |
| | | | apoptosis | | | | |
| UBE2L6 | NM_004223 | Ubiquitin | ubiquitin-conjugating enzyme E2L 6, | 7.7 | 1.8 | 12.3 | 1.5 |
| | | | ligase | | | | |
| USP15 | AF106069 | Ubiquitin | Cysteine protease deubiquitinating | 3.8 | 2.3 | 23.1 | 1.7 |
| | | | enzyme | | | | |
| USP18 | NM_017414 | Ubiquitin | Cysteine protease, ubiquitin specific | 3.8 | 2.4 | 4.1 | 1.9 |
| | | | protease 18 | | | | |
| AB037725 | AB037725 | Other | KIAA1304 | — | — | 23.1 | 1.5 |
| AF086367 | AF086367 | Other | ZD66F04 | — | — | 7.0 | 1.7 |
| AGR2 | NM_006408 | Other | anterior gradient 2 | — | — | 3.0 | 2.1 |
| AK000877 | AK000877 | Other | AJ002784 | — | — | 3.0 | 2.8 |
| AK021733 | AK021733 | Other | HEMBA1004730 | 10.9 | 1.7 | — | — |
| AK021936 | AK021936 | Other | HEMBA1007073 | — | — | 7.0 | 1.7 |
| AL049423 | AL049423 | Other | AL049423 | — | — | 4.1 | 1.7 |
| AL110204 | AL110204 | Other | AL110204 | — | — | 12.3 | 1.6 |
| ATP6B1 | NM_001692 | Other | nucleotide and nucleic acid | — | — | 23.1 | 1.5 |
| | | | transport; Cation transport | | | | |
| B2M | NM_004048 | Other | MHCI-mediated immunity | — | — | 4.1 | 1.7 |
| BG610654 | AK000422 | Other | Beta-galactosidase | — | — | 12.3 | 1.6 |
| BHLHB2 | NM_003670 | Other | mRNA transcription regulation; Cell | — | — | 12.3 | 1.8 |
| | | | proliferation and differentiation | | | | |
| BST2 | NM_004335 | Other | bone marrow stromal cell antigen | 12.8 | 1.7 | 23.1 | 1.5 |
| BTBD2 | NM_017797 | Other | protein-protein interactions, | — | — | 12.3 | 1.6 |
| | | | cytoplasmic bodies | | | | |
| BTG3 | NM_006806 | Other | Cell proliferation and differentiation | — | — | 4.1 | 1.7 |
| C14orf161 | AK024360 | Other | None | — | — | 7.0 | 1.9 |
| C15ORF2 | NM_018958 | Other | cell survival, calcium-sequestering | — | — | 12.3 | 2.0 |
| CBFA1 | AF053952 | Other | OSF2 transcription factor | — | — | 7.0 | 1.8 |
| CHEK2 | NM_007194 | Other | Protein phosphorylation; Stress | — | — | 4.1 | 1.8 |
| | | | response | | | | |
| CITED2 | NM_006079 | Other | Transcription cofactor | — | — | 23.1 | 1.4 |
| CNN1 | NM_001299 | Other | Muscle contraction | — | — | 23.1 | 1.6 |
| D17210 | D17210 | Other | hmd3f02m3 | — | — | 12.3 | 1.6 |
| DAAM1 | AB014566 | Other | Non-motor actin binding protein | — | — | 23.1 | 1.5 |
| DDX58 | NM_014314 | Other | Nucleoside, nucleotide and nucleic | 3.8 | 2.2 | 7.0 | 1.8 |
| | | | acid metabolism; Apoptosis | | | | |
| EGR1 | NM_001964 | Other | mRNA transcription regulation | — | — | 7.0 | 1.9 |
| ENSest7951 | ENSest7951 | Other | ENSestG00000007951 | — | — | 7.0 | 1.7 |
| | AF061034 | Other | TNFA or Fas-ligand pathways | | | | |
| | | | (apoptosis, | | | | |
| | | | inflammation, vasoconstriction) | | | | |
| FIP2 | | | | — | — | 23.1 | 1.4 |
| FLJ11021 | AK001883 | Other | splicing factor, arginine/serine-rich 4 | — | — | 23.1 | 1.5 |
| FLJ20035 | AK001649 | Other | Helicase activity, Nucleic acid binding | — | — | 12.3 | 1.6 |
| FLJ20156 | NM_017691 | Other | Leucine rich repeat | 10.9 | 1.6 | 7.0 | 2.3 |
| FLJ22761 | AK026414 | Other | Hexokinase-1 | — | — | 23.1 | 2.3 |
| FLJ34585 | AK022228 | Other | None | — | — | 23.1 | 1.5 |
| | | Other | Viral oncogene, mRNA transcription | | | | |
| | | | regulation; Immunity and defense; | | | | |
| FOS(1) | NM_005252 | | | — | — | 3.0 | 2.4 |
| | | Other | Viral oncogene, mRNA transcription | | | | |
| | | | regulation; Immunity and defense; | | | | |
| FOS(2) | NM_005252 | | | — | — | 4.1 | 2.0 |
| GADD45A | NM_001924 | Other | DNA repair; Stress | — | — | 12.3 | 1.6 |
| | | | response; Apoptosis; Cell cycle control | | | | |
| GEM | NM_005261 | Other | G-protein mediated signaling; Cell | — | — | 3.0 | 2.0 |
| | | | structure and motility | | | | |
| GENX- | NM_003943 | Other | Genethonin-1, Carbohydrate | — | — | 7.0 | 1.5 |
| 3414 | | | metabolism | | | | |
| GHRGV9A | AF230800 | Other | growth hormone receptor gene, 5′UTR | 10.9 | 2.0 | — | — |
| | | | V9A region | | | | |
| H1F2 | NM_005319 | Other | Chromatin packaging and remodeling | — | — | 7.0 | 1.6 |
| HEY1 | NM_012258 | Other | Basic helix-loop-helix transcription | — | — | 3.0 | 1.9 |
| | | | factor; Nucleic acid binding | | | | |
| HSP70B | NM_002155 | Other | Protein folding; Protein complex | — | — | 12.3 | 1.7 |
| | | | assembly; Stress response | | | | |
| HSPA1B | NM_005346 | Other | Protein folding; Protein complex | — | — | 12.3 | 2.0 |
| | | | assembly; Stress response | | | | |
| HSPF1 | NM_006145 | Other | Protein folding | — | — | 4.1 | 1.8 |
| IER5 | NM_016545 | Other | immediate early response 5, cellular | — | — | 4.1 | 1.7 |
| | | | response to mitogenic signals | | | | |
| IGFBP6 | M62402 | Other | Human insulin-like growth factor | — | — | 12.3 | 1.6 |
| | | | binding protein 6 | | | | |
| ITGB3 | NM_000212 | Other | Cell adhesion-mediated | — | — | 23.1 | 1.5 |
| | | | signaling; Blood clotting; Cell motility | | | | |
| JUN(1) | NM_002228 | Other | mRNA transcription regulation; JNK | — | — | 3.0 | 1.8 |
| | | | cascade; Cell cycle control | | | | |
| JUN(2) | NM_002228 | Other | mRNA transcription regulation; JNK | — | — | 23.1 | 1.6 |
| | | | cascade; Cell cycle control | | | | |
| KIAA0590 | AK023912 | Other | Collagen alpha-1(I) chain precursor | — | — | 23.1 | 1.4 |
| KIAA1404 | AK023836 | Other | Transcription factor activity | — | — | 23.1 | 1.8 |
| KLF6(1) | AL117595 | Other | Transcriptional activator activity, B-cell | — | — | 23.1 | 1.5 |
| | | | growth and development | | | | |
| KLF6(2) | NM_001300 | Other | Transcriptional activator activity, B-cell | — | — | 23.1 | 1.6 |
| | | | growth and development | | | | |
| KRT17 | NM_000422 | Other | Intermediate filament; Structural protein | 5.4 | 1.9 | 3.0 | 3.8 |
| KYNU | NM_003937 | Other | Amino acid metabolism | — | — | 23.1 | 1.7 |
| LBA1 | AB002340 | Other | belongs to the ribosomal protein s12p | — | — | 7.0 | 1.7 |
| | | | family | | | | |
| LGP2 | AK021416 | Other | Nucleoside, nucleotide and nucleic | 3.8 | 2.6 | 3.0 | 2.6 |
| | | | acid metabolism | | | | |
| LOC283737 | AL133446 | Other | lysosome, degradation of dermatan | — | — | 23.1 | 1.5 |
| | | | and keratan sulfates. | | | | |
| LOC93082 | AL389981 | Other | ortholog of mouse lung-inducible | 5.4 | 2.3 | 7.0 | 5.1 |
| | | | C3HC4 RING domain protein | | | | |
| LPIN1 | D80010 | Other | Lipid metabolism; Developmental | — | — | 4.1 | 1.9 |
| | | | processes | | | | |
| LRRN3 | AL442092 | Other | Leucine rich repeat neuronal 3 | — | — | 7.0 | 1.6 |
| LY6E(1) | NM_002346 | Other | lymphocyte antigen 6 complex, locus | 5.4 | 2.4 | 3.0 | 2.1 |
| | | | E; LY6E | | | | |
| LY6E(2) | NM_002346 | Other | lymphocyte antigen 6 complex, locus | 5.4 | 1.9 | 12.3 | 1.8 |
| | | | E; LY6E | | | | |
| MGC40405 | AB046797 | Other | Zinc finger, SWIM-type containing 6 | — | — | 23.1 | 1.6 |
| MGC45731 | AK027019 | Other | Zinc finger protein 697, transcription | — | — | 7.0 | 1.7 |
| | | | factor | | | | |
| MKP-1(1) | AJ227912 | Interferon | Response to oxidative stress | — | — | 3.0 | 1.9 |
| MKP-1(2) | NM_004417 | Interferon | Response to oxidative stress | — | — | 3.0 | 1.9 |
| PARP14 | AB033094 | Other | Protein amino acid ADP-ribosylation | 5.4 | 1.9 | 4.1 | 2.2 |
| PLK2 | NM_006622 | Other | Protein phosphorylation; Intracellular | — | — | 12.3 | 1.5 |
| | | | signaling cascade | | | | |
| pLSB8 | U03241 | Other | Clone pLSB8 chromosome 21 STS | — | — | 23.1 | 1.7 |
| PMAIP1 | D90070 | Other | Oncogenesis | — | — | 3.0 | 2.8 |
| PTTG1 | NM_004219 | Other | DNA repair; mRNA transcription | — | — | 23.1 | 1.6 |
| | | | regulation; Cell cycle control | | | | |
| RAB27A | NM_004580 | Other | Receptor mediated | — | — | 23.1 | 1.4 |
| | | | endocytosis; General vesicle transport | | | | |
| REC8L1 | NM_005132 | Other | chromatid cohesion phosphoprotein of | 18.8 | 1.5 | — | — |
| | | | the rad21p family | | | | |
| RGS2 | NM_002923 | Other | G-protein mediated signaling | — | — | 12.3 | 1.7 |
| RHO6 | NM_014470 | Other | G-protein mediated signaling; Cell | — | — | 12.3 | 2.0 |
| | | | structure and motility | | | | |
| RRAD | NM_004165 | Other | GTPase activity TAS GOA/IPI | 3.8 | 3.0 | 7.0 | 1.9 |
| SDCBP | NM_005625 | Other | GTPase mediated signal transduction | — | — | 23.1 | 1.5 |
| SHB | NM_003028 | Other | SHB adaptor protein (a Src homology 2 | — | — | 12.3 | 1.5 |
| | | | protein) (SHB) | | | | |
| SPAG9 | NM_003971 | Other | Intracellular signaling | — | — | 12.3 | 1.6 |
| | | | cascade; Transport; Cell structure and | | | | |
| | | | motility | | | | |
| SUMO2 | NM_006937 | Other | Protein modification; Inhibition of | — | — | 7.0 | 1.9 |
| | | | apoptosis | | | | |
| TES | NM_015641 | Other | Actin binding cytoskeletal | — | — | 23.1 | 1.4 |
| | | | protein; Structural protein | | | | |
| TNIP1 | NM_006058 | Other | Nef-associated factor 1 | — | — | 12.3 | 1.6 |
| TOP1 | J03250 | Other | DNA unwinding | — | — | 12.3 | 1.8 |
| TSPYL2 | AF273046 | Other | DNA replication; Chromatin packaging | — | — | 23.1 | 1.7 |
| | | | and remodeling; Apoptosis | | | | |
| TULP3 | NM_003324 | Other | tubby like protein 3 | — | — | 4.1 | 1.7 |
| VIP | NM_003381 | Other | Regulation of vasoconstriction, dilation | — | — | 12.3 | 1.9 |
| WBP5 | NM_016303 | Other | Transcription factor | — | — | 7.0 | 1.5 |
| ZC3HAV1 | NM_020119 | Other | Zinc finger CCCH type antiviral protein 1 | — | — | 12.3 | 1.7 |

### Example 9

### Expression Analysis: TaqMan Low Density Array (TLDA)

In order to confirm and validate the microarray results, fifty nine genes identified by microarray and 36 genes selected by pathway analysis are investigated further using a quantitative TaqMan low density array (TLDA).

In the HepG2 model, at 48 and 72 hrs, 31 and 62 genes respectively are confirmed to be differentially expressed by TLDA (Table E4 below). In the A549 infection model, TLDA revealed a higher number of differentially expressed genes, with 63 at the 48 hrs and 82 at 72 hrs (Table E4 below). In Singapore dengue fever patients, TLDA analysis of blood samples taken during acute febrile stage (day 1), compared to samples at convalescence (Day 21), revealed 67 differentially expressed genes (Table E4 below).

| | Accession | HepG2 | | A549 | | | |
|---|---|---|---|---|---|---|---|
| Gene Name | number | 48 hrs | 72 hrs | 48 hrs | 72 hrs | Patients | P-value |
| ABCA1 | NM_005502 | 2.7 | −4.2 | 4.9 | 1.8 | 13.3 | 1.4E−06 |
| ATF3 | NM_004024 | 3.3 | 1.6 | 1.6 | 8.7 | 26.7 | 2.3-06 |
| ATP6V1B1 | NM_001692 | −41.5 | 1.0 | −1.1 | −1.8 | 1.0 | |
| B2M | NM_004048 | 1.6 | 1.3 | 2.4 | 7.3 | 2.2 | 7.4E−12 |
| BCL2L10 | NM_020396 | 1.0 | 1.0 | 1.0 | 1.0 | 1.0 | |
| BTG3 | NM_006806 | 4.0 | 1.6 | 2.1 | 3.8 | 3.0 | 3.0E−04 |
| CAMK2B | U23460 | 1.0 | 1.0 | 1.0 | 1.0 | 1.0 | |
| CBL | NM_005188 | 2.8 | −1.2 | 1.6 | −1.6 | 1.1 | 8.4E−03 |
| RANTES | NM_002985 | 296.5 | 1079.5 | 69.0 | 88.6 | −1.7 | |
| CHEK2 | NM_007194 | −1.2 | 1.6 | 3.8 | 1.0 | 2.9 | 2.7E−02 |
| CITED2 | NM_006079 | 1.9 | 2.1 | 1.9 | 2.5 | 1.3 | 1.7E−04 |
| CNN1 | NM_001299 | −86.8 | −1.0 | 1.0 | 1.0 | 1.0 | |
| IP-10 | NM_001565 | 69.3 | 94.2 | 63.7 | 85.3 | 323.5 | 1.1E−05 |
| I-TAC | NM_005409 | 325.9 | 402.3 | 305.1 | 814.3 | 801.5 | 2.1E−06 |
| CXCR4 | NM_001008540 | −1.7 | −20.4 | 79.7 | 1.0 | 2.3 | |
| RIG-I | NM_014314 | 32.9 | 3.1 | 5.3 | 21.6 | 5.5 | 1.9E−09 |
| DHRS2 | NM_005794 | 1.2 | 1.0 | 3.6 | 1.9 | 1.0 | |
| DNAJB1 | NM_006145 | 1.3 | 2.0 | −1.4 | −1.5 | 1.1 | 1.7E−04 |
| MKP-1 | NM_004417 | 1.9 | 2.2 | 1.0 | −1.4 | 2.4 | 2.0E−07 |
| EGR1 | NM_001964 | 1.8 | 2.0 | 31.4 | 6.7 | 2.5 | 7.1E−05 |
| EIF2AK2 | NM_002759 | 2.6 | 2.0 | 1.5 | 1.5 | 4.6 | 2.3E−08 |
| ERN1 | NM_001433 | 1.3 | 1.2 | 2.6 | 1.6 | −1.6 | |
| FGD3 | AK000004 | 117.0 | −2.0 | 1.0 | −160.3 | 1.4 | |
| ISG15 | NM_005101 | 16.2 | 10.4 | 6.2 | 12.2 | 16.8 | 2.0E−09 |
| G1P3 | NM_002038 | 28.2 | 18.3 | 1.7 | 2.9 | 6.5 | 2.2E−10 |
| GADD45A | NM_001924 | 1.2 | 1.7 | 2.8 | 1.1 | 2.5 | 9.8E−05 |
| GEM | NM_005261 | 1.0 | 115.6 | 1.1 | 2.3 | 1.0 | |
| GENX | NM_003943 | −1.9 | 8.8 | −1.4 | 1.2 | 9.4 | 5.8E−05 |
| HERC1 | U50078 | 1.0 | −1.9 | −2.2 | −6.3 | −1.1 | |
| HERC2 | AF071172 | 1.3 | −1.0 | −1.1 | −1.2 | −1.3 | |
| HERC3 | D25215 | 1.0 | 268.9 | −2.9 | −42.9 | −1.6 | |
| HERC4 | NM_015601 | 1.2 | −1.3 | −1.1 | −1.4 | 1.1 | |
| HERC5 | NM_016323 | 399.4 | 681.8 | 11.8 | 14.2 | 6.5 | 5.4E−10 |
| HERPUD1 | NM_014685 | 1.4 | 1.7 | 2.2 | −1.2 | 1.2 | 1.7E−03 |
| HEY1 | NM_012258 | 171.7 | 6.3 | 83.9 | −287.8 | −29.1 | |
| HIST1H1C | NM_005319 | −1.1 | −1.2 | −2.1 | −1.6 | 1.1 | |
| HNRPK | NM_002140 | −1.1 | 1.1 | 1.0 | 1.1 | 1.8 | 5.6E−04 |
| HSPA1L | NM_005346 | 9.9 | 1.6 | −2.9 | 58.9 | −1.6 | |
| HSPA6 | NM_002155 | 110.5 | 3.3 | 1.0 | 1.0 | 2.8 | |
| IER3 | NM_003897 | 1.5 | 1.0 | 2.3 | 1.9 | −1.4 | |
| IFI44 | NM_006417 | 244.0 | 139.1 | 2.9 | 148.5 | 6.2 | 8.9E−13 |
| MDA5 | AF095844 | 16.4 | 11.1 | 10.7 | 22.8 | 8.9 | 5.2E−12 |
| IFNA1 | NM_024013 | 1.0 | 1.0 | −56.3 | −205.1 | 1.0 | |
| IFNB1 | NM_002176 | 224.7 | 1.9 | 21.0 | 1684.3 | −2.3 | |
| IFNG | NM_000619 | 1.0 | 1.0 | 1.0 | 1.0 | 6.7 | |
| IFRG28 | AJ251832 | 55.2 | 56.7 | 267.1 | 4.7 | 8.1 | 1.6E−13 |
| IGFBP6 | M62402 | 679.3 | 12.1 | −1.1 | 1.3 | 1.0 | |
| IL10 | NM_000572 | 1.0 | 1.0 | 1.0 | 1.0 | 9.6 | |
| IL2 | NM_000586 | 1.0 | 1.0 | 1.0 | 1.0 | 1.0 | |
| IL3RA | NM_002183 | 1.0 | 1.0 | 1.0 | 1.0 | 6.3 | |
| IL6 | NM_000600 | 1.0 | 1.0 | 342.7 | 733.1 | 58.9 | |
| IL8 | M17017 | 16.9 | 4.9 | 9.7 | 17.7 | −1.0 | |
| IRF9 | NM_006084 | 3.9 | 2.4 | 1.8 | 1.4 | 1.9 | 2.6E−08 |
| ITCH | NM_031483 | 1.6 | −1.2 | 2.3 | −1.3 | 1.4 | |
| ITGB3 | NM_000212 | 1.0 | 1.0 | 58.5 | 223.8 | 1.9 | |
| KRT17 | NM_000422 | 635.0 | 7.3 | −1.2 | 2.1 | 1.0 | |
| KYNU | NM_003937 | −1.4 | 2.0 | 2.0 | 2.3 | 3.4 | 1.0E−06 |
| LGALS1 | NM_002305 | 1.0 | −1.3 | −1.2 | −2.0 | 1.9 | |
| MAFB | NM_005461 | 1.0 | 42.5 | 1.0 | 1.0 | 4.2 | |
| Hdm2 | NM_002392 | 1.0 | 1.4 | 1.2 | 1.9 | 1.6 | 3.6E−03 |
| MPL | NM_005373 | 1.0 | 1.0 | 1.0 | 1.0 | −1.7 | |
| MxA | NM_002462 | 2285.7 | 7.6 | 5.0 | 8.6 | 9.8 | 5.9E−14 |
| NEDD4 | NM_006154 | 1.1 | −2.2 | 1.1 | 1.8 | 2.3 | |
| NFKBIA | NM_020529 | 1.9 | 2.3 | 3.9 | 6.4 | 2.5 | 3.7E−11 |
| NFKBIB | NM_002503 | 1.1 | 1.5 | 1.7 | 1.7 | 2.0 | 1.8E−05 |
| INOS | NM_000625 | 1.0 | 1.0 | 1.0 | 59.3 | 1.0 | |
| OAS1 | NM_016816 | 18.9 | 26.8 | 4.0 | 4.0 | 6.1 | 8.1E−11 |
| OAS2 | NM_002535 | 464.4 | 218.5 | 8.5 | 39.4 | 6.8 | 2.8E−11 |
| OAS3 | NM_006187 | 92.9 | 2.3 | 3.8 | 4.6 | 6.2 | 3.6E−13 |
| OASL | AF063611 | 2878.8 | 6132.2 | 9.1 | 57.2 | 7.8 | 3.8E−12 |
| PLK2 | NM_006622 | 1.6 | 2.3 | 1.4 | 1.0 | 48.7 | 4.6E−04 |
| PSMB9 | NM_002800 | 207.8 | 137.2 | 13.3 | 23.3 | 2.8 | 1.1E−11 |
| COX2 | NM_000963 | 1.0 | 1.0 | 5.4 | 4.3 | −1.3 | |
| RAB27A | NM_004580 | 1476.3 | 106.8 | 7.1 | 4.3 | 1.1 | 3.1E−04 |
| RGS2 | NM_002923 | 1.0 | 18.3 | 1.3 | 1.0 | −2.1 | |
| RHO6 | NM_014470 | 10.3 | 8.0 | 2.0 | 1.8 | 41.2 | 8.5E−06 |
| RRAD | NM_004165 | 1.3 | 2.8 | −27.7 | 3.2 | 46.2 | 5.2E−04 |
| Viperin | AF026941 | 431.7 | 917.4 | 19.0 | 2264.3 | 11.2 | 2.1E−10 |
| PAI1 | NM_000602 | 1.3 | 1.3 | 2.4 | 5.6 | 3.5 | 1.4E−05 |
| SOCS1 | NM_003745 | 2.0 | −1.3 | 4.3 | 20.9 | 21.3 | 1.1E−06 |
| STAT1 | NM_007315 | 4.2 | 2.1 | 2.5 | 5.7 | 3.3 | 4.5E−13 |
| TGFB1 | NM_000660 | 1.0 | −1.3 | 1.1 | −1.2 | 1.5 | |
| TNFAIP3 | NM_006290 | 1.3 | 2.9 | 10.4 | 16.0 | 3.3 | 6.1E−08 |
| TNF | NM_000594 | 1.0 | 13.4 | 1.0 | 284.8 | 2.6 | 1.7E−04 |
| TNFRSF11B | NM_000881 | 1.0 | 1.0 | 1.0 | 1.0 | 1.0 | |
| TNIP1 | NM_006058 | 1.3 | 1.3 | 2.9 | 5.4 | 1.1 | 2.4E−07 |
| TULP3 | NM_003324 | 1.6 | 4.1 | 1.0 | −1.4 | −4.9 | |
| UBB | NM_018955 | 1.2 | −1.2 | 1.1 | −1.1 | −1.2 | |
| UBE1C | NM_003968 | 2.7 | 1.1 | 1.4 | 1.4 | 1.4 | 9.5E−04 |
| UBE2I | NM_003345 | 1.4 | −1.4 | 1.0 | −1.3 | 1.2 | |
| UBE2L6 | NM_004223 | 1.6 | −1.2 | 5.4 | 7.5 | 4.7 | |
| USP15 | AF106069 | 3.4 | −1.3 | −1.1 | 3.1 | 1.5 | 6.8E−05 |
| USP18 | NM_017414 | 3.6 | 20.8 | 4.2 | 5.7 | 31.0 | 9.9E−13 |
| VEGF | NM_003376 | 1.3 | 1.0 | 1.2 | 1.8 | 1.3 | 5.5E−04 |
| VIP | NM_003381 | 1.0 | 42.0 | 1.0 | 1.0 | 1.0 | |

Table E4. Fold increase in gene expression as determined by TLDA. Cell lines HepG2 and A549 are fold change during dengue virus infection over stimulation with heat-inactivated virus. EDEN samples are patient samples at peak of fever (Day 1) over those from the same patients at convalescence (Day 21). Up-regulation is shown in black and down-regulation or no significant change is shown in red (significance determined by q value<5). Genes that are significantly up-regulated in at least one time point in HepG2 and in at least one time point in A549 and in the single time point in Patients are indicated with P-values, calculated by SAM analysis. Differentially expressed genes are selected using a procedure known as Significance Analysis of Microarrays (SAM), described in brief below: The statistic used in SAM is given as

$$d = \frac{{μ}_{1} - {μ}_{2}}{s + {s}_{0}}$$

where; the numerator is the group mean difference, s the standard error, and so a regularizing constant. Setting so=0 will yield a t-statistic. This value, called the fudge constant, is found by removing the trend in d as a function of s in moving windows across the data to reduce false positive results. As the statistic is not t-distributed significance is computed using a permutation test. Genes with a computed statistic larger than the threshold are called significant. The false discovery rate (FDR) associated with the given threshold can also be calculated from the permutation data.

### Example 10

### Expression Analysis: 50 Up-Regulated Genes

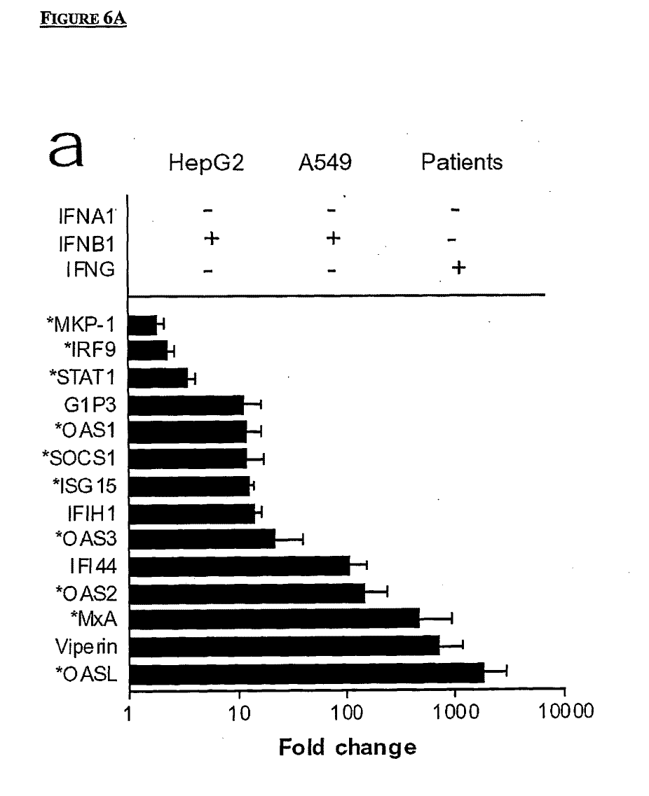

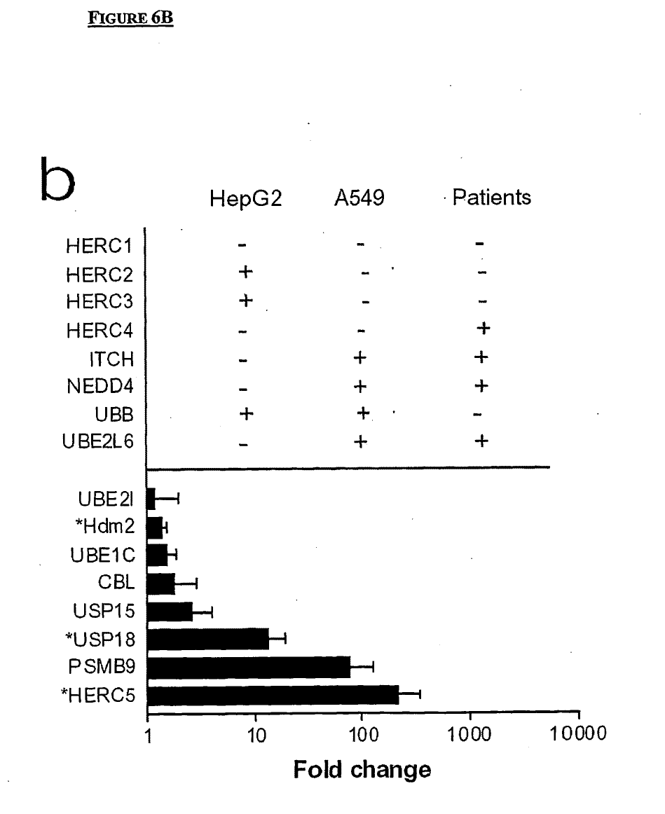

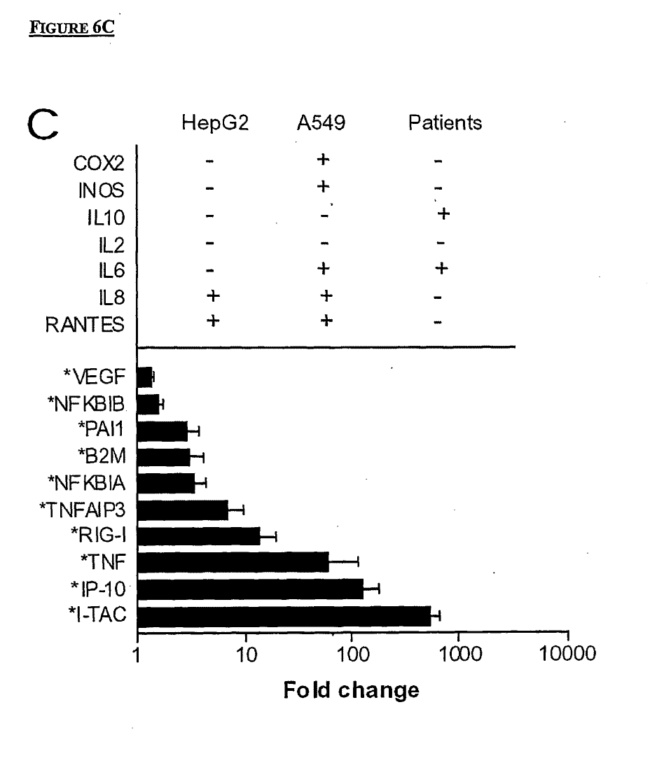

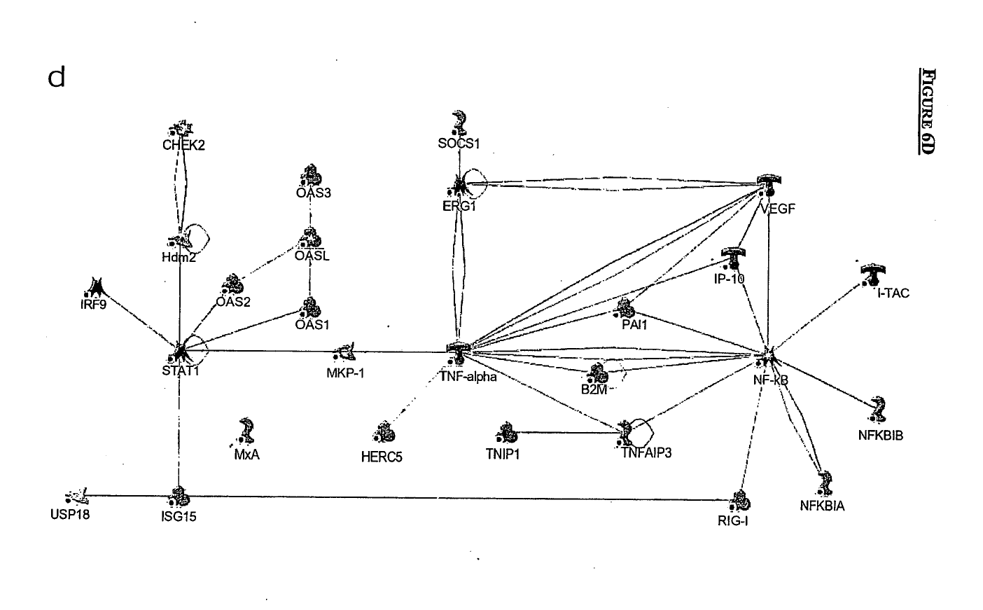

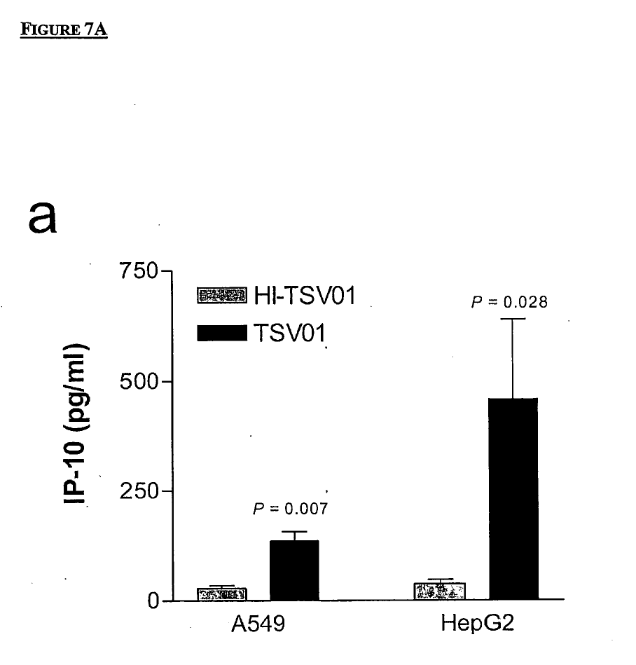

We then selected those genes from the TLDA results that are up-regulated in at least one time point in HepG2 cells and in at least one time point in A549 cells and in the single time point in patient samples and combined them to identify a list of 50 common genes that are significantly up-regulated in all three systems (Table E4, FIG. 6).

Among the fifty genes, a large number of them identify with three biological processes: the interferon-related genes (FIG. 6A), the ubiquitin-proteasome system genes (FIG. 6B) and the NF-κB -mediated cytokine/chemokine response genes (FIG. 6C), even though these three pathways are not mutually exclusive

The mean fold increase (±s.e.m, n=3-5) of each gene in all three systems is pooled and listed from the highest to the lowest up-regulation in each group (FIGS. 6A, 6B and 6C). Genes confirmed by TLDA to be not up-regulated in all three systems are also shown as comparison (FIGS. 6A, 6B and 6C). Of interest is the differential cytokine response in each system; IL-8, and RANTES are up-regulated in cell lines but not patient samples, while IL-10 is up-regulated in patient samples but not cell lines, indicative of the effects of multiple cells types in vivo.

These fifty common genes are further mapped by direct interactions using the MetaCore program which illustrated the close clustering and interconnection of a network of 29 of the genes around NF-κB, TNF-α and interferon response genes. (FIG. 6D). The NF-κB gene alone is added to the list of common genes to illustrate the connections between those genes induced by it.

The 50 genes comprise the following: interferon-mediated genes: IFNA1, IFNB1, IFNG, MKP-1, IRF9, STAT1, G1P3, OAS1, SOCS1, ISG15, IFIH1, OAS3, IF144, OAS2, MxA, Viperin and OASL; ubiquitin-proteasome system genes: HERC1, HERC2, HERC3, HERC4, ITCH, NEDD4, UBB, UBE2L6, UBE2I, Hdm2, UBE1C, CBL, USP15, USP18, PSMB9 and HERC5; and NF-κB-mediated cytokine/chemokine response genes: COX2, INOS, IL10, IL2, IL6, IL8, RANTES, VEGF, NFKBIB, PAI1, B2M, NFKBIA, TNFAIP3, RIG-I, TNF, IP-10 and I-TAC.

### Example 11

### Expression Analysis: Cytokines/mediators/IP-10 and I-TAC

The two most highly up-regulated cytokines/mediators in each of the three cell systems are IP-10 and I-TAC. The production of these related chemokines leads to the recruitment of CXCR3 expressing T cells (Loetscher, Gerber et al. 1996; Cole, Strick et al. 1998) and they have a negative association with a small number of infections including SARS (Tang, Chan et al. 2005) and HCV (Helbig, Ruszkiewicz et al. 2004).

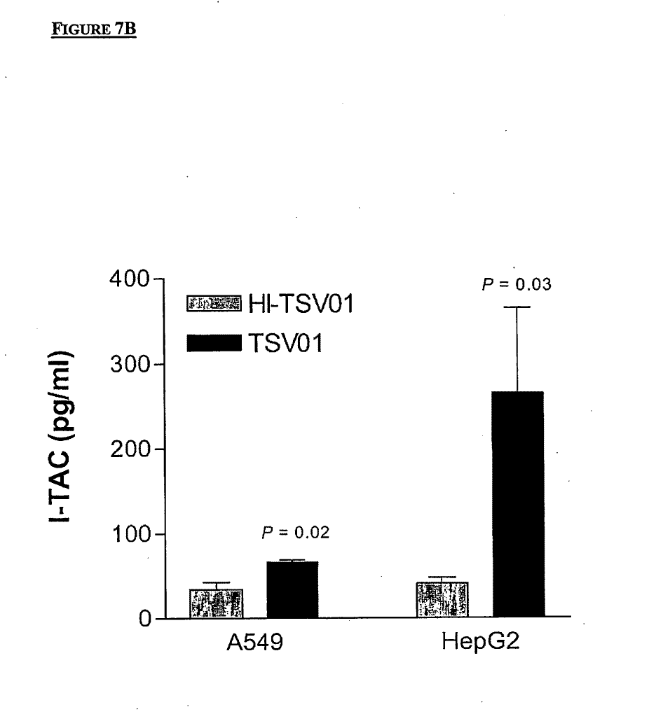

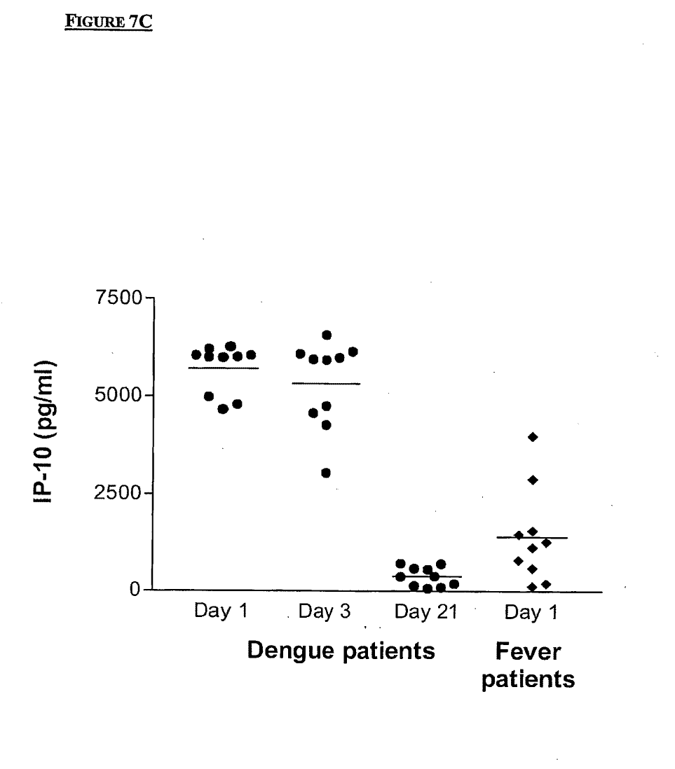

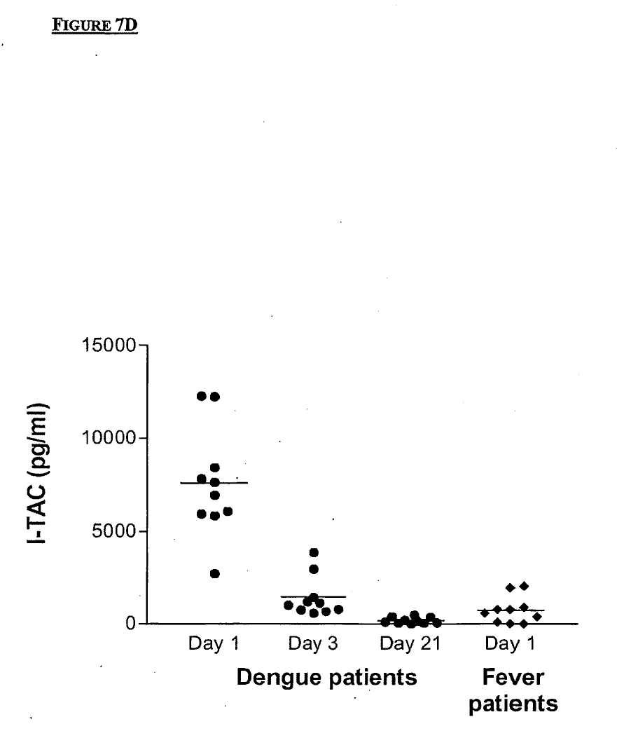

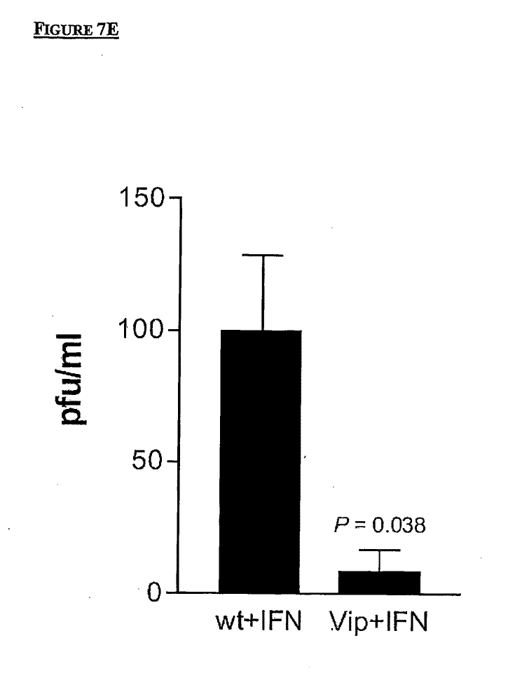

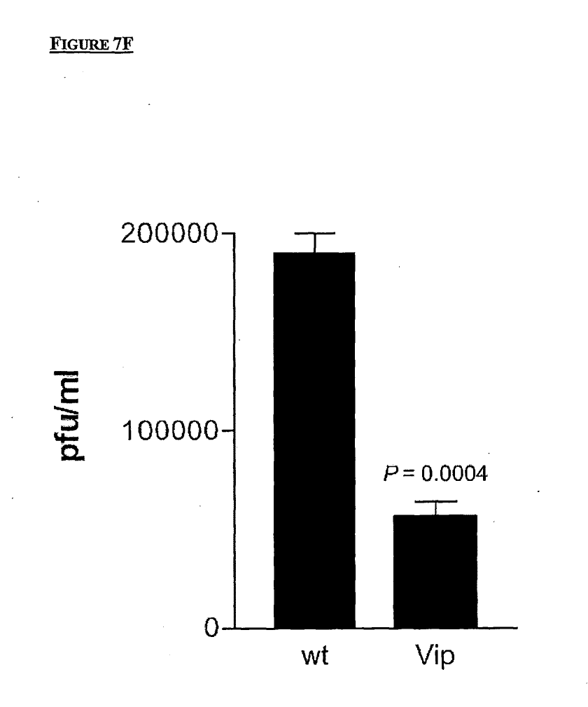

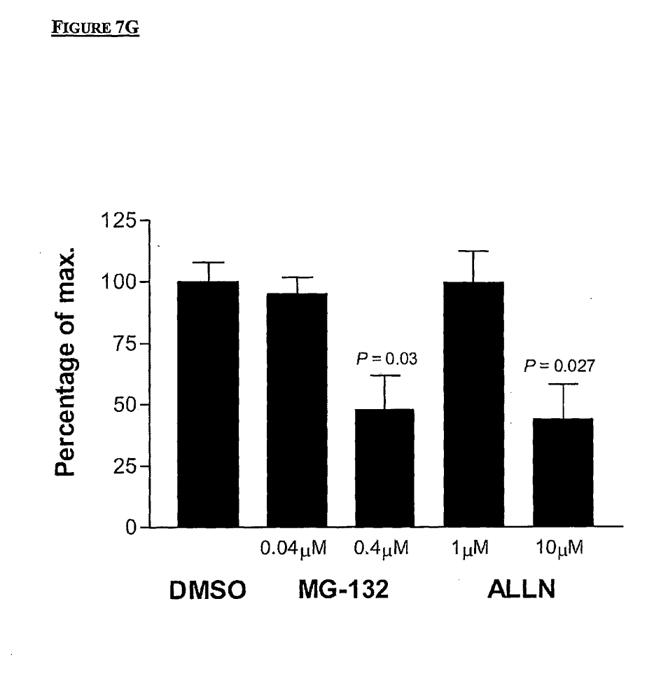

The A549 and HepG2 infection models produced moderate concentrations of IP-10 (FIG. 7A) and I-TAC (FIG. 7B) Patient serum samples are assayed for IP-10 (FIG. 7C) and I-TAC (FIG. 7D). There is significantly more P-10 in the serum of day 1 (P=10⁻¹⁵) and day 3 (P=10⁻¹¹) dengue patients comparing to the convalescent serum as well as to non dengue related fever patient serum (day 1 P=10⁻⁹, day 3 P=10⁻⁷). I-TAC level is also significantly higher in day 1 dengue patient serum comparing to both the convalescent (P=10⁻⁷) and non dengue related fever (P=10⁻⁶).

These results suggest a specificity for IP-10 and I-TAC for dengue infection. It may be that concentrations of T-10 and I-TAC can be used as a marker for dengue fever.

Both IP-10 and I-TAC are induced by NF-κB activation and the effect of NF-κB on virus replication is determined by adding dexamethasone to the HepG2 infection model (Auphan, DiDonato et al. 1995). Dexamethasone inhibited IP-10 and I-TAC production, but had no effect on viral replication (data not shown). These results suggest that NF-κB activation and IP-10 and I-TAC production have no direct effect on viral replication in vitro.

### Example 12

### Expression Analysis: Interferon/Viperin

The ability of interferon pre-treatment to inhibit subsequent dengue replication has been previously reported (Diamond, Roberts et al. 2000), as has the importance of interferon in the anti-viral response (Simmen, Singh et al. 2001).

One of the most highly upregulated common genes from the interferon-related pathway is viperin, previously identified as an interferon-induced, anti-viral protein in HCMV and HCV infection (Chin and Cresswell 2001): Over-expression of viperin in the A549 cell line (Vip) resulted in a significant reduction in viral replication compared to wild-type control cells (wt) as shown by plaque assay two days after infection with (FIG. 7E) and without (FIG. 7F) pre-treatment with IFN-β (500 U/ml).

Although the greatest anti-viral effect is achieved by the combination of pre-treatment with IFN-β in Viperin overexpressing cells, Viperin overexpression alone resulted in significant reduction of virus production (P=0.0004) (FIG. 7F). These results suggest that viperin is but part of the interferon-mediated response to dengue, and demonstrate for the first time that viperin by itself could be an important component of the anti-dengue response.

### Example 13

### Expression Analysis: Ubiquitination/MG-132 and ALLN Inhibitors

Ubiquitination is a key component of the immune system, the conjugation of single or multiple ubiquitin molecules to a protein targets it for trafficking or for destruction in the proteasome (reviewed in (Liu, Penninger et al. 2005)).

Components of the ubiquitin-proteasome system have been shown to be required for the maturation and release of the retroviruses, Rous sarcoma virus and HIV (Patnaik, Chau et al. 2000; Schubert, Ott et al. 2000; Stack, Calistri et al. 2000). The ubiquitin-proteasome and interferon systems are interconnected, for example HERC5 is shown to be induced by interferon and required for conjugation of ISG15 (Dastur, Beaudenon et al. 2006).

Inhibition of the ubiquitin-proteasome system in the HepG2 infection model, using specific proteasome inhibitors MG-132 and ALLN, significantly and consistently reduced virus replication in the cell lines as shown by plaque assay (FIG. 7G). Further studies may elucidate the precise role the ubiquitin-proteasome plays in dengue replication, possibly virus trafficking or maturation and fusion upon release (Kuhn, Zhang et al. 2002).

### REFERENCES

Auphan, N., J. A. DiDonato, et al. (1995). “Immunosuppression by glucocorticoids: inhibition of NF-kappa B activity through induction of I kappa B synthesis.” Science 270(5234): 286-90.

Chin, K. C. and P. Cresswell (2001). “Viperin (cig5), an IFN-inducible antiviral protein directly induced by human cytomegalovirus.” Proc Natl Acad Sci U S A 98(26): 15125-30.

Cole, K. E., C. A. Strick, et al. (1998). “Interferon-inducible T cell alpha chemoattractant (I-TAC): a novel non-ELR CXC chemokine with potent activity on activated T cells through selective high affinity binding to CXCR3.” J Exp Med 187(12): 2009-21.

Dastur, A., S. Beaudenon, et al. (2006). “HercS, an interferon-induced HECT E3 enzyme, is required for conjugation of ISG15 in human cells.” J Biol Chem 281(7): 4334-8.

Diamond, M. S., T. G. Roberts, et al. (2000). “Modulation of Dengue virus infection in human cells by alpha, beta, and gamma interferons.” J Virol 74(11): 4957-66.

Helbig, K. J., A. Ruszkiewicz, et al. (2004). “Expression of the CXCR3 ligand I-TAC by hepatocytes in chronic hepatitis C and its correlation with hepatic inflammation.” Hepatology 39(5): 1220-9.

Jenner, R. G. and R. A. Young (2005). “Insights into host responses against pathogens from transcriptional profiling.” Nat Rev Microbiol 3(4): 281-94.

Jessie, K., M. Y. Fong, et al. (2004). “Localization of dengue virus in naturally infected human tissues, by immunohistochemistry and in situ hybridization.” J Infect Dis 189(8): 1411-8.

Kuhn, R. J., W. Zhang, et al. (2002). “Structure of dengue virus: implications for flavivirus organization, maturation, and fusion.” Cell 108(5): 717-25.

Liu, Y. C., J. Penninger, et al. (2005). “Immunity by ubiquitylation: a reversible process of modification.” Nat Rev Immunol 5(12): 941-52.

Loetscher, M., B. Gerber, et al. (1996). “Chemokine receptor specific for IP10 and mig: structure, function, and expression in activated T-lymphocytes.” J Exp Med 184(3): 963-9.

Matsuda T, Almasan A, Tomita M, Tamaki K, Saito M, Tadano M, Yagita H, Ohta T, Mori N. Dengue virus-induced apoptosis in hepatic cells is partly mediated by Apo2 ligand/tumour necrosis factor-related apoptosis-inducing ligand. J Gen Virol. 2005 April; 86(Pt 4):1055-65.

McBride W, Vasudevan S. 1995. Relationship of a dengue 2 isolate from Townsville, 1993, to international isolates. Commun Dis Intell(19):522-523.

McBride, W. J. H. and S. G. Vasudevan (1995). “Relationship of a dengue 2 isolate from Townsville, 1993, to international isolates.” Communicable Diseases Intelligence 19: 522-523.

Moses, A. V., M. A. Jarvis, et al. (2002). “Kaposi's sarcoma-associated herpesvirus-induced upregulation of the c-kit proto-oncogene, as identified by gene expression profiling, is essential for the transformation of endothelial cells.” J Virol 76(16): 8383-99.

Patnaik, A., V. Chau, et al. (2000). “Ubiquitin is part of the retrovirus budding machinery.” Proc Natl Acad Sci U S A 97(24): 13069-74.

Rigau-Perez, J. G., G. G. Clark, et al. (1998). “Dengue and dengue haemorrhagic fever.” Lancet 352(9132): 971-7.

Schubert, U., D. E. Ott, et al. (2000). “Proteasome inhibition interferes with gag polyprotein processing, release, and maturation of HIV-1 and HIV-2.” Proc Natl Acad Sci U S A 97(24): 13057-62.

Simmen, K. A., J. Singh, et al. (2001). “Global modulation of cellular transcription by human cytomegalovirus is initiated by viral glycoprotein B.” Proc Natl Acad Sci U S A 98(13): 7140-5.

Strack, B., A. Calistri, et al. (2000). “A role for ubiquitin ligase recruitment in retrovirus release.” Proc Natl Acad Sci U S A 97(24): 13063-8.

Tang, N. L., P. K. Chan, et al. (2005). “Early enhanced expression of interferon-inducible protein-10 (CXCL-10) and other chemokines predicts adverse outcome in severe acute respiratory syndrome.” Clin Chem 51(12): 2333-40.

Vaughn D W, Green S, Kalayanarooj S, Innis B L, Nimmannitya S, Suntayakorn S, Endy T P, Raengsakulrach B, Rothman A L, Ennis F A, Nisalak A. 2000. Dengue viremia titer, antibody response pattern, and virus serotype correlate with disease severity. J Infect Dis 181(1):2-9.

Vaughn, D. W., S. Green, et al. (2000). “Dengue viremia titer, antibody response pattern, and virus serotype correlate with disease severity.” J Infect Dis 181(1): 2-9.

Zhang Y, Hwang H M, Ekunwe S. Comparing cytotoxicity and genotoxicity in HaCaT cells caused by 6-aminochrysene and 5,6-chrysenequinone under ultraviolet A irradiation. Environ Toxicol Chem. 2006 July; 25(7):1920-5.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of providing an indication useful in the diagnosis or prognosis of dengue, the method comprising detecting a change in the expression pattern or level of: (a) a ubiquitin-proteasome pathway protein; (b) a interferon-related protein; or (c) an NF-κB-mediated cytokine/chemokine response protein.

2. A method of treatment or prevention of dengue in an individual, the method comprising modulating the level of expression of: (a) a ubiquitin-proteasome pathway protein; or (b) a interferon-related protein.

3. A method of identifying a molecule suitable for the treatment of dengue, the method comprising determining if a candidate molecule is an agonist or antagonist of (a) a ubiquitin-proteasome pathway protein; or (b) a interferon-related protein.

4. A method according to Claim 3, in which the candidate molecule is exposed to (a) a ubiquitin-proteasome pathway protein; or (b) a interferon-related protein in order to determine if the candidate molecule is an agonist or antagonist thereof

5. (canceled)

6. A method for providing an indication useful in the diagnosis or prognosis of dengue, the method comprising detecting a polymorphism in (a) a ubiquitin-proteasome pathway protein; (b) a interferon-related protein; or (c)an NF-κB-mediated cytokine/chemokine response protein, in a sample from the individual.

7. A method of identifying an agonist or antagonist of: (a) a ubiquitin-proteasome pathway protein; or (b) a interferon-related protein, the method comprising exposing the candidate molecule to a cell infected with dengue virus and determining an effect on viral function.

8. A method according to Claim 7, in which the viral function is selected from the group consisting of: viral titre, viral infectivity, viral replication, viral packaging and viral transcription.

9. A method of identifying an agonist or antagonist of: (a) a ubiquitin-proteasome pathway protein; or (b) a interferon-related protein, the method comprising administering a candidate molecule to an animal suffering from dengue and determining whether the animal exhibits a decrease or increase in dengue virus replication.

10. (canceled)

11. A method of down-regulating a dengue viral function in a cell infected with dengue virus, the method comprising modulating the activity of (a) a ubiquitin-proteasome pathway protein; or (b) a interferon-related protein in the cell.

12. A method according to Claim 11, in which the viral function is selected from the group consisting of: viral titre, viral infectivity, viral replication, viral packaging and viral transcription.

13. (canceled)

14. (canceled)

15. A method according to claim 1, in which the ubiquitin-proteasome pathway protein is selected from the group consisting of: a ubiquitin specific protease, a ubiquitin-conjugating enzyme, a ubiquitin ligase and a ubiquitin cleavage enzyme.

16. A method according to claim 15, in which the ubiquitin-proteasome pathway protein is selected from the group consisting of: HERC1 (U50078), HERC2 (AF071172), HERC3 (D25215), HERC4 (NM_{—}015601), C17orf27 (AB046774), DTX3L (AK025135), HERC6 (NM_{—}017912), RNF36 (AL360161), ITCH (NM_{—}031483), NEDD4 (NM_{—}006154), UBB (NM_{—}018955), UBE2L6 (NM_{—}004223), UBE2I (NM_{—}003345), Hdm2 (NM_{—}002392), UBE1C (NM_{—}003968), CBL (NM_{—}005188), USP15 (AF106069), USP18 (NM_{—}017414), PSMB9 (NM_{—}002800), UBE2 (NM_{—}003335), UBP43 (NM_{—}017414), HERC5 (NM_{—}016323), ATG7 (NM_{—}006395), DUSP1 (NM_{—}004417.2), DUSP18 (NM_{—}152511.2), DUSP3 (NM_{—}004090.2), DUSP5 (NM_{—}004419.2), EIF3S5 (NM_{—}003754), PPP1R15A (NM_{—}014330.2), PSMB8 (NM_{—}148919), UBE1L (NM_{—}003335), UBE2L6 (NM_{—}004223), UBE2S (NM_{—}014501), UBE2W (NM_{—}018299), USP24 (XM_{—}165973.4) and WWP1 (NM_{—}007013).

17. A method according to claim 15, in which the ubiquitin-proteasome pathway protein comprises ubiquitin specific protease 18 (USP18, GenBank Accession Number: NM_{—}017414) or Ubiquitin-conjugating enzyme E2L (UBE2L6, GenBank Accession Number: NM_{—}004223).

18. A method according to claim 15, in which the ubiquitin-proteasome pathway protein inhibitor comprises MG-132 (Carbobenzoxy-L-leucyl-L-leucyl-L-leucinal) or ALLN (N-Acetyl-Leu-Leu-Nle-CHO).

19. (canceled)

20. (canceled)

21. A method according to claim 1, in which the interferon-mediated protein is selected from the group consisting of: IFNA1 (NM_{—}024013), IFNB1 (NM_{—}002176), IFNG (NM_{—}000619), ATF3 (NM_{—}004024) MKP-1 (NM_{—}004417, AJ227912), IRF9 (NM_{—}006084), STAT1 (AK022231, NM_{—}007315), G1P2 (NM_{—}005101), G1P3 (NM_{—}002038), IF144 (NM_{—}006417), IFIT1 (NM_{—}001548), IFIT2 (AF026944), IFIT3 (AF026943), ISGF3G (NM_{—}006084), IER3 (NM_{—}003897), IFIT5 (NM_{—}012420), IFRG28 (AJ251832), MDA5 (AF095844), SP110 (NM_{—}004510), STAT1 (NM_{—}007315), OAS1 (NM_{—}016816), SOCS1 (NM_{—}003745), ISG15 (NM_{—}005101), IFIH1 (AL080107), OAS3 (NM_{—}006187), IF144 (NM_{—}006417), OAS2 (NM_{—}002535), MxA (NM_{—}002462), Viperin (AF026941, AF026942), OASL (AF063611), GBP1 (NM_{—}002053), IRF1 (NM_{—}002198), IRF7 (NM_{—}004030), GBP2 (NM_{—}004120), NMI (NM_{—}004688), AIM2 (NM_{—}004833), STAT2 (NM_{—}005419), IF116 (NM_{—}005531), SLAMF7 (NM_{—}021181), GBP4 (NM_{—}052941) and GBP5 (NM_{—}052942).

22. A method according to claim 21, in which the interferon-mediated protein comprises viperin (GenBank Accession Number: AF026941, AF026942) or interferon alpha (IFN-α, GenBank Accession Number: NM_{—}024013).

23. A method of down-regulating a dengue viral function, for example viral titre, viral infectivity, viral replication, viral packaging or viral transcription, in a cell infected with dengue virus, the method comprising up-regulating the activity of viperin (GenBank Accession Number: AF026941, AF026942) or interferon alpha (IFN-α, GenBank Accession Number: NM_{—}024013) in the cell.

24. A method according to claim 23, further comprising up-regulating the activity of IFN-β (GenBank Accession Number: NM_{—}002176) in the cell.

25. (canceled)

26. A method according to claim 1, in which the polypeptide comprises an NF-κB-mediated cytokine/chemokine response protein.

27. A method according to claim 26, in which the NF-κB-mediated cytokine/chemokine response protein is selected from the group consisting of: COX2 (NM_{—}000963), INOS (NM_{—}000625), IL10 (NM_{—}000572), IL2 (NM_{—}000586), IL6 (NM_{—}000600), IL8 (M17017), RANTES (NM_{—}002985), VEGF (NM_{—}003376), NFKBIB (NM_{—}002503), PAI1 (NM_{—}000602), B2M (NM_{—}004048), NFKBIA (NM_{—}020529), TNFAIP3 (NM_{—}006290), RIG-I (NM_{—}014314), TNF (NM_{—}000594), CCL4 (NM_{—}002984), CCL5 (NM_{—}002985), IL11b (NM_{—}000881), IP-10 (NM_{—}001565), I-TAC (NM_{—}005409), CARD15 (NM_{—}022162), CARD4 (NM_{—}006092), CD14 (NM_{—}000591), CD1A (NM_{—}001763), CD2 (NM_{—}001767), CD22 (NM_{—}001771), CD276 (NM_{—}025240), CD47 (NM_{—}001777), CD59 (NM_{—}000611), CD97 (NM_{—}001784), CCL2 (NM_{—}002982), CCR1 (NM_{—}001295), CCR5 (NM_{—}000579), CCR7 (NM_{—}001838), CCRL2 (NM_{—}003965), CXCL16 (NM_{—}022059), IL1RN (NM_{—}173842), IL10RB (NM_{—}000628), IL13RA1 (NM_{—}001560), IL16 (NM_{—}004513), IL18 (NM_{—}001562), IL18RAP (NM_{—}003853), IL4R (NM_{—}000418), IL8RA (NM_{—}000634), IL8RB (NM_{—}001557), PF4 (NM_{—}002619), PBEF1 (NM_{—}182790), TNFSF10 (NM_{—}003810), TNFRSF1A (NM_{—}001065), TNFRSF1B (NM_{—}001066), TNFRSF25, (NM_{—}148970), TNFRSF7 (NM_{—}001242), TNFAIP2 (NM_{—}006291) and TNFAIP8 (NM_{—}014350).

28. A method according to claim 26, in which the NF-κB-mediated cytokine/chemokine response protein comprises IP-10 (GenBank Accession Number: NM_{—}001565).

29. A method according to claim 26, in which the NF-κB-mediated cytokine/chemokine response protein comprises I-TAC (GenBank Accession Number: NM_{—}005409).

30. A method of providing an indication useful in the diagnosis or prognosis of dengue, the method comprising detecting a change in the expression pattern or level of any one or more of the following: interferon alpha (IFN-α, GenBank Accession Number: NM_{—}024013), IP-10 (GenBank Accession Number: NM_{—}001565) or I-TAC (GenBank Accession Number: NM_{—}005409).

31. A kit for diagnosis or prognosis of dengue, the kit comprising means for the detection of a change in the expression pattern or level of any one or more of the following: (a) a ubiquitin-proteasome pathway protein; (b) a interferon-related protein; or (c) an NF-κB-mediated cytokine/chemokine response protein, together with instructions for use.

32. A kit for treatment or prevention of dengue in an individual, the kit comprising means for modulating the level of expression of: (a) a ubiquitin-proteasome pathway protein; or (b) a interferon-related protein, together with instructions for use.

33. A kit according to claim 32, comprising MG-132 (Carbobenzoxy-L-leucyl-L-leucyl-L-leucinal) or ALLN (N-Acetyl-Leu-Leu-Nle-CHO) or both.

34. A kit according to claim 32, further comprising any one or more of the following: P4-PMO compounds 5′SL and 3′CS, any of the fullerenes described in U.S. Pat. No. 6,777,445 and Helioxanthin and/ or an analogue thereof (U.S. Pat. No. 6,306,899)

35-39. (canceled)


SEQUENCE LISTING (1 sequence)

SEQ ID NO: 1 - protein, 4 aa, Homo sapiens
DEAD